SEQUENCE LISTING (68 sequences)

SEQ ID NO: 1 - DNA, 18 bp, Artificial Sequence
  PR1RT
taggatggttagggggtg

SEQ ID NO: 2 - DNA, 18 bp, Artificial Sequence
  PR2RT
gagcatgtcctcaaactt

SEQ ID NO: 3 - DNA, 18 bp, Artificial Sequence
  PR3RT
cacattccctatcccgaa

SEQ ID NO: 4 - DNA, 18 bp, Artificial Sequence
  PR4RT
ctaattgaataggtgact

SEQ ID NO: 5 - DNA, 32 bp, Artificial Sequence
  PR1Fz
gatggccgtgggggccccctttaaccatgtct

SEQ ID NO: 6 - DNA, 27 bp, Artificial Sequence
  PR1Rz
gatttaattaacaaagcaaccaggtaa

SEQ ID NO: 7 - DNA, 32 bp, Artificial Sequence
  PR2Fz
gatggccgtgggggccccggatatcgtcgcgg

SEQ ID NO: 8 - DNA, 27 bp, Artificial Sequence
  PR2Rz
gatttaattaaccatatgctgtgcata

SEQ ID NO: 9 - DNA, 32 bp, Artificial Sequence
  PR3Fz
gatggccgtgggggccaagctttccgtggagc

SEQ ID NO: 10 - DNA, 27 bp, Artificial Sequence
  PR3Rz
gatttaattaagtgtagctgaaggaca

SEQ ID NO: 11 - DNA, 32 bp, Artificial Sequence
  PR4Fz
gatggccgtgggggccattatgaggggaagaa

SEQ ID NO: 12 - DNA, 27 bp, Artificial Sequence
  PR4Rz
gatttaattaaacgcggatcaggcgca

SEQ ID NO: 13 - DNA, 18 bp, Artificial Sequence
  PR50
aaaagtcttcaggcttgg

SEQ ID NO: 14 - DNA, 27 bp, Artificial Sequence
  PRX
ccagtgttgtggcctgcagggcgaatt

SEQ ID NO: 15 - DNA, 18 bp, Artificial Sequence
  PR49
cgacccgtaccattcttt

SEQ ID NO: 16 - DNA, 27 bp, Artificial Sequence
  PRXR
gatgaattcgccctgcaggccacaaca

SEQ ID NO: 17 - DNA, 21 bp, Artificial Sequence
  PR41
tgcttgaccgcgggctgttgc

SEQ ID NO: 18 - DNA, 15465 bp, PRRSV PL97-1/PL1 virus sequence
atgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggttgctttggcaccataaggtgtcaaccctgattgatgaaatggtgtcgcgtcgaatgtaccgcatcatggaaaaatcagggcaggctgcctggaaacaggtggtgagcgaggctacgctgtctcgcattagtagtttggatgtggtggctcattttcagcatctagccgccattgaagccgagacctgtaaatatttggcctcccgactgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttactatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaa

SEQ ID NO: 19 - DNA, 24 bp, Artificial Sequence
  PR40
agctgtacattcctccatattttc

SEQ ID NO: 20 - DNA, 24 bp, Artificial Sequence
  PRcR
ggtttgcaggcgaacgcctgagaa

SEQ ID NO: 21 - DNA, 24 bp, Artificial Sequence
  PRcF
gttcgcctgcaaaccatgtttcag

SEQ ID NO: 22 - DNA, 18 bp, Artificial Sequence
  JP41
tccgtggaatgaacaatg

SEQ ID NO: 23 - DNA, 30 bp, Artificial Sequence
  PRI5Rsp6
acacctatacgtcatctatagtgtccccta

SEQ ID NO: 24 - DNA, 18 bp, Artificial Sequence
  PRI1F
atgacgtataggtgttgg

SEQ ID NO: 25 - DNA, 31 bp, Artificial Sequence
  PR3endX
cgttgcggccgccctgcaggttaattaagat

SEQ ID NO: 26 - DNA, 25 bp, Artificial Sequence
  PR3endY
atctctagattaattaacctgcagg

SEQ ID NO: 27 - DNA, 23218 bp, pBAC/PRRSV/FL
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggttgctttggcaccataaggtgtcaaccctgattgatgaaatggtgtcgcgtcgaatgtaccgcatcatggaaaaatcagggcaggctgcctggaaacaggtggtgagcgaggctacgctgtctcgcattagtagtttggatgtggtggctcattttcagcatctagccgccattgaagccgagacctgtaaatatttggcctcccgactgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttactatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 28 - DNA, 18 bp, Artificial Sequence
  PR3364
ccattcctgcgtagcagg

SEQ ID NO: 29 - DNA, 21 bp, Artificial Sequence
  PR2172
gccacgcgtaatcgatacctt

SEQ ID NO: 30 - DNA, 23218 bp, pBAC/PRRSV/FLgm
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgataccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggttgctttggcaccataaggtgtcaaccctgattgatgaaatggtgtcgcgtcgaatgtaccgcatcatggaaaaatcagggcaggctgcctggaaacaggtggtgagcgaggctacgctgtctcgcattagtagtttggatgtggtggctcattttcagcatctagccgccattgaagccgagacctgtaaatatttggcctcccgactgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttactatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 31 - DNA, 18 bp, Artificial Sequence
  PR1282
ccattggctgacaaggaa

SEQ ID NO: 32 - DNA, 23155 bp, pBAC/PRRSV/FLnop(A)
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggttgctttggcaccataaggtgtcaaccctgattgatgaaatggtgtcgcgtcgaatgtaccgcatcatggaaaaatcagggcaggctgcctggaaacaggtggtgagcgaggctacgctgtctcgcattagtagtttggatgtggtggctcattttcagcatctagccgccattgaagccgagacctgtaaatatttggcctcccgactgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttactatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattctagagcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 33 - DNA, 18 bp, Artificial Sequence
  PR37
gatagggacacccgtgta

SEQ ID NO: 34 - DNA, 31 bp, Artificial Sequence
  PRnop(A)
agggcggccgctctagaatttcggccgcatg

SEQ ID NO: 35 - DNA, 24057 bp, pBAC/PRRSV/RepLuc MB
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggttgctttggcaccataaggtgtcaaccctgattgatgaaatggtgtcgcgtcgaatgtaccgcatcatggaaaaatcagggcaggctgcctggaaacaggtggtgagcgaggctacgctgtctcgcattagtagtttggatgtggtggctcattttcagcatctagccgccattgaagccgagacctgtaaatatttggcctcccgactgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtaccttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 36 - DNA, 22449 bp, pBAC/PRRSV/RepLuc DI
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 37 - DNA, 23506 bp, pBAC/PRRSV/RepLuc ME
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 38 - DNA, 26 bp, Artificial Sequence
  PRdiR
gatgagctccagggcggccgcaacgt

SEQ ID NO: 39 - DNA, 26 bp, Artificial Sequence
  PRs1
tatctgcagtagtttgcctacgcatc

SEQ ID NO: 40 - DNA, 26 bp, Artificial Sequence
  PRs2
tatctgcaggggacttgcaccctgtc

SEQ ID NO: 41 - DNA, 26 bp, Artificial Sequence
  PRs3
tatctgcagagcggcaattgtgtctg

SEQ ID NO: 42 - DNA, 26 bp, Artificial Sequence
  PRs4
tatctgcagttttcctctagcgactg

SEQ ID NO: 43 - DNA, 26 bp, Artificial Sequence
  PRs5
tatctgcagggcaagggaccgggaaa

SEQ ID NO: 44 - DNA, 24 bp, Artificial Sequence
  PRs6
tatctgcagagctgtgccagatgc

SEQ ID NO: 45 - DNA, 24 bp, Artificial Sequence
  PRs7
tatctgcagcaacggcaagcagca

SEQ ID NO: 46 - DNA, 24 bp, Artificial Sequence
  PRs8
tatctgcagagggcttctgcacct

SEQ ID NO: 47 - DNA, 22506 bp, pBAC/PRRSV/RepLuc S1
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcagtagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 48 - DNA, 22556 bp, pBAC/PRRSV/RepLuc S2
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcaggggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 49 - DNA, 22606 bp, pBAC/PRRSV/RepLuc S3
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcagagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 50 - DNA, 22656 bp, pBAC/PRRSV/RepLuc S4
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcagttttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 51 - DNA, 22706 bp, pBAC/PRRSV/RepLuc S5
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcagggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 52 - DNA, 22756 bp, pBAC/PRRSV/RepLuc S6
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcagagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 53 - DNA, 22806 bp, pBAC/PRRSV/RepLuc S7
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcagcaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 54 - DNA, 23206 bp, pBAC/PRRSV/RepLuc S8
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatcttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaatgaatgcagagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 55 - DNA, 27 bp, Artificial Sequence
  O7-5
tgctctagaatggtgagcaagggcgag

SEQ ID NO: 56 - DNA, 27 bp, Artificial Sequence
  O7-6
cctaagcttattgagctcgagatctga

SEQ ID NO: 57 - DNA, 27 bp, Artificial Sequence
  O7-2
catagatctcttctgctgcttgccgtt

SEQ ID NO: 58 - DNA, 24901 bp, pBAC/PRRSV/FL/Npro-EGFP
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggttgctttggcaccataaggtgtcaaccctgattgatgaaatggtgtcgcgtcgaatgtaccgcatcatggaaaaatcagggcaggctgcctggaaacaggtggtgagcgaggctacgctgtctcgcattagtagtttggatgtggtggctcattttcagcatctagccgccattgaagccgagacctgtaaatatttggcctcccgactgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttactatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagatctatggagttgatcacaaatgaacttttatacaaaacatacaaacaaaaacccgtcggggtggaggaacctgtttatgatcaggcaggtgatcccttatttggtgaaaggggagcagtccaccctcaatcgacgctaaagctcccacacaagagaggggaacgcgatgttccaaccaacttggcatccttaccaaaaagaggtgactgcaggtcgggtaatagcagaggacctgtgagcgggatctacctgaagccagggccactattttaccaggactataaaggtcccgtctatcacagggccccgctggagctctttgaggagggatccatgtgtgaaacgactaaacggatagggagagtaactggaagtgacggaaagctgtaccacatttatgtgtgtatagatggatgtataataataaaaagtgccacgagaagttaccaaagggtgttcaggtgggtccataataggcttgactgccctctatgggtcacaagttgctctagaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaataagctgagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 59 - DNA, 27 bp, Artificial Sequence
  O7-1
gatctcgagacgcgtgtaccagatata

SEQ ID NO: 60 - DNA, 27 bp, Artificial Sequence
  O7-3
aagagatctatggagttgatcacaaat

SEQ ID NO: 61 - DNA, 27 bp, Artificial Sequence
  O7-4
cattctagagcaacttgtgacccatag

SEQ ID NO: 62 - DNA, 24 bp, Artificial Sequence
  O7-7
aataagcttagggcttctgcacct

SEQ ID NO: 63 - DNA, 25 bp, Artificial Sequence
  O7-8
atcccatgggcggccgcaacgtttt

SEQ ID NO: 64 - DNA, 25054 bp, pBAC/PRRSV/FL/IRES-EGFP
gatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggttgctttggcaccataaggtgtcaaccctgattgatgaaatggtgtcgcgtcgaatgtaccgcatcatggaaaaatcagggcaggctgcctggaaacaggtggtgagcgaggctacgctgtctcgcattagtagtttggatgtggtggctcattttcagcatctagccgccattgaagccgagacctgtaaatatttggcctcccgactgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttactatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagatccttgatccaggaagacagggtcatctagtgtgacttaaggtagaaatgtaaataatgtgaatgagaaaacgaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataagcttgccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgagctcaataagctgagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcaggcatgccctcgtccacgtggcatctcgagacctttattccaaggcgtcgaaccactgacgactaccctgtactcagggcttaagccatccaacgaactcaccactgttgctacccccctcattatgctagtcctactaagggcatggctagcctcttttcggccttcgctgagagggatttgttccctaggcctaattattatttttaattgcccaatacgtatacgagtgccttttctaattctcgtatactatagtgagtcgtattatctagccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgtgtctcaaaatctctgatgttacattgcacaagataaaaatatatcatcatgaacaataaaactgtctgcttacataaacagtaatacaaggggtgttatgagccatattcaacgggaaacgtcttgctcgacgatgataagctgtcaaacatgagaattgggtcgtcaatatgctaaaacgcggcataccccgcgtattcccactagttaattaacctgcagggggctgttagaggtcttccctagtccaactatagcgtatggacatattgtcgttagaacgcggctacaattaatacataaccttatgtatcatacacatacgatttaggggacactata

SEQ ID NO: 65 - DNA, 24 bp, PRorf7F
gattctagaatgccaaataacaac

SEQ ID NO: 66 - DNA, 29 bp, Artificial Sequence
  PRorf7R
atcgtttaaacttatcatgctgagggtga

SEQ ID NO: 67 - DNA, 11234 bp, pSinRep19/PRRSV ORF7
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacctaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggattcatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaataccacaacaccaccacctctagaatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgataagtttgtgagcatgcaggattacacctgtcctactggcattgagaacttttgcccagagcaaaagagcattccaagccatcagaggggaaataaagcatctctacggtggtcctaaatagtcagcatagtacatttcatctgactaatactacaacaccaccacctctagctagagcttaccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatgatcgctagaccatggggtaccgagctcgaattcgcctcgtcgctattaattataggacttatgatttttgcttgcagcatgatgctgactagcacacgaagatgacgggcccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttcaaaaaaaaaaaaaaaaaaaaaaaaaaaaaaaaaaaaagggaattcctcgattaattaagcggccgctcgaggggaattaattcttgaagacgaaagggccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgagctcgtatggacatattgtcgttagaacgcggctacaattaatacataaccttatgtatcatacacatacgatttaggggacactatag

SEQ ID NO: 68 - DNA, 15505 bp, PRRSV/FL
gacactatagatgacgtataggtgttggctctatgccttggcatttgtattgtcaggtgctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttatggcggagggccaagtctactgcacacgatgcctcagtgcacggtctctccttcccctgaacctccaagtttctgagctcggggtgctaggcctattctacaggcccgaagagccactccggtggacgttgccacgtgcattccccactgttgagtgctcccccgccggggcctgctggctttctgcaatctttccaatcgcacgaatgaccagtggaaacctgaacttccaacaaagaatggtacgggtcgcagctgagctttacagagccggccagctcacccctgcagtcttgaaggctctacaagtttatgaacggggttgccgctggtaccccattgttggacctgttcctggagtggccgttttcgccaattccctacatgtgagtgataaacctttcccgggagcaactcacgtgttgaccaacctgccgctcccgcagagacccaagcctgaagacttttgcccctttgagtgtgctatggctactgtctatgacattggtcatgacgccgtcatgtatgtggccgaaaggaaaatctcctgggcccctcgtggcggggatgaagtgaaatttgaagctgtccccggggagttgaagttgattgcgaaccggctccgcacctccttcccgccccaccacacagtggacatgtctaagttcgccttcacagcccctgggtgtggtgtttctatgcgggtcgaacgccaacacggctgccttcccgctgacactgtccctgaaggcaactgctggtggagcttgtttgacttgcttccactggaagttcagaacaaagaaattcgccatgctaaccaatttggctaccagaccaagcatggtgtctctggcaagtacctacagcggaggctgcaagttaatggtctccgagcagtaactgacccaaacggacctatcgtcgtacagtacttctccgttaaggagagttggatccgccatttgaaactggcgggagaacccagctaccctgggtttgaggacctcctcagaataagggttgagcataacacgtcgccattggctgacaaggaagaaaaaattttccggtttggcagtcacaagtggtacggcgctggaaagagagcaagaaaagcacgctcttgtgcgactgctacagtcgctggccgcgctttgtccgttcgtgaaacccggcaggccaaggagcacgaggttgccggcgccaacaaggctgagcacctcaaacactactccccgcctgccgaagggaattgtggttggcactgcatttccgccatcgccaaccggatggtgaattccaaatttgaaaccacccttcccgaaagagtgagacctccagatgactgggctactgacgaggatcttgtgaatgccatccaaatcctcagactccctgcggccttagacaggaacggtgcttgtactagcgccaagtacgtacttaagctggaaggtgagcattggactgtcactgtgacccctgggatgtccccttctttgctccctcttgaatgtgttcagggctgttgtgggcacaagggcggtcttggttccccagatgcagtcgaggtctccggatttgaccctgcctgccttgaccggctggctgaggtgatgcacctgcctagcagtgctatcccagccgctctggccgaaatgtctggcgattccgatcgttcggcttctccggtcaccaccgtgtggactgtttcgcagttctttgcccgtcatagcggagggaatcaccctgaccaagtgcgcttagggaaaattatcagcctttgtcaggtgattgaggactgctgctgttcccagaacaaaactaaccgggtcaccccggaggaggtcgcagcaaagattgacctgtacctccgtggtgcaacaaatcttgaagaatgcttggccaggcttgagaaagcgcgcccgccacgcgtaatcgacaccttctttgattgggatgttgtgctccctggggttgaggcggcaacccagacgatcaagctgccccaggtcaaccagtgtcgtgctctggtccctgttgtgactcaaaagtccttggacaacaactcggtccccctgaccgccttttcactggctaaccactactaccgtgcgcaaggtgacgaagttcgtcaccgtgaaagactaaccgccgtgctctccaatttggaaaaggttgtccgagaagaatatgggctcatgccaaccgagcctggtccacggcccacactgccacgcgggctcgacgaactcaaagaccagatggaggaggacttgctgaaactggctaacgcccagacgacttcggacatgatggcctgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgccccgcgcaggaaggttgggtccgattgtggcagcccggtttcattaggcggcgatgtccctaacagttgggaagatttggctgttagtagcccctttgatctcccgaccccacctgagccggcaacaccttcaagtgagctggtgattgtgtcctcaccgcaatgcatcttcaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcaatcccaccgtaccagaacgagcccctggatttgtctgcttcctcacagactgaatatgaggcctctcccccagcaccgccgcagagtgggggcgttctgggagtagaggggcatgaagctgaggaaaccctgagtgaaatctcggacatgtcgggtaacattaaacctgcgtccgtgtcatcaagcagctccttgtccagcgtgagaatcacacgcccaaaatactcagctcaagccatcatcgactcgggcgggccctgcagtgggcatctccaagaggtaaaggaagcatgccttagtgtcatgcgcgaggcatgtgatgcgactaagcttgatgaccctgctacgcaggaatggctttctcgcatgtgggatcgggtggacatgctgacttggcgcaacacgtctgtttaccaggcgatttgcaccttagatggcaggttaaagttcctcccaaaaatgatactcgagacaccgccgccctatccgtgtgagtttgtgatgatgcctcacacgcctgcaccttccgtaggtgcggagagcgaccttaccattggctcagttgctactgaagatgttccacgcatcctcgagaaaatagaaaatgtcggcgagatggccaaccagggacccttggccttctccgaggataaaccggtagatgaccaacttgtcaacgacccccggatatcgtcgcggaggcctgacgagagcacatcagctccgtccgcaggcacaggtggcgccggcttttttaccgatttgccgccttcagatggcgcggatgcggacgggggggggccgtttcggacggtaaaaagaaaagctgaaaggctctttgaccaactgagccgtcaggtttttgacctcgtctcccatctccctgttttcttctcacgccttttctgccctggcggtggttattctccgggtgattggggttttgcagctcttactctattgtgcctctttttatgttacagttacccagcctttggtattgctcccctcttgggtgtgttttctgggtcttctcggcgcgttcgaatgggggtttttggctgctggttggcttttgctgttggtctgttcaagcctgtgtccgacccagtcggcgctgcttgtgagtttgactcgccagagtgtagaaacatccttcattcttttgagcttctcaaaccttgggaccctgttcgcagccttgtcgtgggccccgtcggtctcggtcttgccattcttggcaggttactgggcggggcacgctgcatctggcactttttgcttaggcttggcattgttgcagactgtatcttggctggagcttacgtgctttctcaaggtaggtgtaaaaagtgctggggatcttgtataagaactgctcctaatgaggtcgcttttaacgtgtttcctttcacacgtgcgaccaggtcgtcacttatcgacctgtgcgatcggttttgtgcgccaaaaggaatggaccccatttttctcgccactgggtggcgcgggtgctgggccggccgaagccccattgagcaaccctctgaaaaacccatcgcgtttgcccaattggatgaaaagaagattacggctaggactgtggtcgcccagccttatgaccccaaccaagccgtaaagtgcttgcgggtattgcaggcgggtggggcgatggtggctaaggcggtcccaaaagtggtcaaggtttccgctgttccattccgagcccccttctttcccactggagtgaaagttgaccctgattgcagggtcgtggttgaccctgacactttcactgcagcactccggtctggctactccaccacaaacctcgtccttggtgtaggggactttgcccagctgaatggattaaaaatcaggcaaatttccaagccttcagggggaggcccacatctcatggctgccctgcatgttgcctgctcgatggctctgcacatgcttgctgggatttatgtgactgcggtgggttcttgcggcaccggcaccaacgacccgtggtgcgctaacccgtttgccgtccctggctacggacctggctctctctgcacgtccagattgtgcatttcccaacacggccttaccctgcccttgacagcacttgtggcgggattcggtattcaagaaattgccttggtcgttttgatttttgtttccatcggaggcatggctcataggttgagctgtaaggctgacatgctgtgtgttttgcttgcaattgccagctatgtttgggtacctcttacctggttgctttgtgtgtttccttgctggttgcgctgtttttctttgcaccccctaaccatcctatggttggtgtttttcttgatttctgtgaatatgccttcaggaatcttggccatggtgttgttggtttctctttggcttcttggtcgttatactaatgttgctggcattgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaagtcaagtgcgtaactgccgcacatgtccttacgggcaattcagctcgggtttccggggtcggcttcaatcaaatgcttgactttgacgtaaagggagatttcgctatggctgattgcccgaattggcaaggggctgcccccaagacccaattctgcacggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaagttctgatgatcaggcttctaacagcagctcttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccatatccttgttggcgatggagtgttctctgcggctttcttcttgagatactttgccgagggaaagttgagggaaggggtgtcgcaatcctgcggaatgaatcatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggtactttggccaaacttgaagcttttgctgataccgtggcacctcaactctcgcccggtgacattgttgtcgctctcggccacacgcctgttggcagtgtcttcgacttaaaggttggtagcaccaagcataccctccaagccattgagaccagagtccttgctgggtccaaaatgaccgtggcgcgcgtcgtcgacccgacccccacgcccccacccgcacccgtgcccatccccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataataacacagatgagtgggagtgtctcagagttggcgaccctgccgactttgaccctgagaagggaactctgtgtggacatgtcaccattgaaaacaaggcttaccatgtttacacctccccatctggtaagaagttcttggtccccgtcaacccagagaatggaagagttcagtgggaagctgcaaagctttccgtggagcaggccctaggtatgatgaatgtcgacggcgaactgactgccaaagaactggagaaactgaagagaataattgacaaactccagggcctgactaaggagcagtgtttaaactgctagccgccagcgacttgacccgctgtggtcgcggcggcttggttgttactgaaacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggcgttgtgctcctgcgttccgcggttccttcgcttatagacgtcttgatctccggtgctgatgcatctcccaagttacttgcccatcacgggccgggaaacactgggatcgatggcacgctctgggattttgagtccgaagccactaaagaggaagtcgcactcagtgcgcaaataatacaggcttgtgacattaggcgcggcgacgctcctgaaattggtctcccttacaagctgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgatgggcgctccgtcttggccacgaccatgccccccgggtttgagttatatgtaccgaccataccagcgtctgtccttgattaccttgactctaggcctgactgccctaaacagctgacagagcacggctgcgaagatgccgcactgaaagacctctctaaatatgacttgtccacccaaggctttgttttacccggagttcttcgccttgtgcggaaatacctgtttgcccatgtaggtaagtgcccacccgttcatcggccttctacttaccctgctaagaattctatggctggaataaatgggaacaggttcccaaccaaggacattcagagcgtccctgaaatcgacgttctgtgcgcacaggctgtgcgagaaaactggcaaactgtcaccccttgtactcttaagaaacagtattgcgggaagaagaagactaggaccatactcggcaccaataacttcatcgcactagcccaccgagcagtgttgagtggtgttacccagggcttcatgaaaaaggcgtttaactcgcccatcgccctcggaaagaacaagtttaaggagctacagactccggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcagtgactaagagaggtggcctgtcgtctggcgacccgatcacctctgtgtctaacaccatttatagtttggtgatctatgcacagcatatggtgcttagttacttcaaaagtggtcacccccatggccttctgttcttacaagaccagctaaagtttgaggacatgctcaaggttcaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagtaatgtttctgaatactatgcctcagcggctgcaatactcatggacagctgtgcttgtttggagtatgatcctgaatggtttgaagaacttgtagttggaatagcgcagtgcgcccgcaaggacggctacagctttcccggcacgccgttcttcatgtccatgtgggaaaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggccatccagcgggttctggttcttgtagtgagtgcaaatcccctgtagggaagggcacaagccctttagacgaggtgctggaacaagtcccgtataagcccccacggaccgttattatgcatgtggagcagggtctcaccccccttgatccaggtagataccaaactcgccgcggactagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttggggacgtgccggttcaacgtcccggcaggcacaacgctgcaattccccgtcccctcccgcaccggcccgtgggttcgcatcctagccggtggttggtgtcctggcaagaattccttcctagatgaagcagcgtattgcaatcaccttgatgttttgaggcttcttagtaaaactaccctcacctgtctaggagacttcaagcaactccacccagtgggttttgattctcattgctatgtttttgacatcatgcctcaaactcaactgaagaccatctggaggtttggacagaatatctgtgatgccattcagccagattacagggacaaactcatgtccatggtcaacacaacccgtgtgacctacgtggaaaaacctgtcaggtatgggcaggtcctcaccccctaccacagggaccgagaggacgacgccatcactattgactccagtcaaggcgccacattcgatgtgattacattgcatttgcccactaaagattcactcaacaggcaaagagcccttgttgccatcaccagggcaagacacgctatctttgtgtatgacccacacaggcagctgcagggcttgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgtgttgtagattctctccgcgccatttgtgctgatctagaagggtcgagctctccgctccccaaggtcgcacacaacttgggatttcatttctcacctgatttaacacagtttgctaaactcccagtagaacttgcacctcactggcccgtggtgacaacccagaacaatgaaaagtggccagatcggctggttgccagccttcgccctatccataaatacagccgcgcgtgcatcggtgctggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgctttcattggcgacgtcaaaggcactaccgttggaggatgtcatcatgtcacctccggatacctcccgcgcgtccttcctaaggaatcagttgcggtagtcggggtttcaagccccggaaaagccgcgaaagcattgtgcacactgacagatgtgtacctcccagatcttgaagcctatctccatccggagacccagtccaagtgctggaaaatgatgttggacttcaaagaagttcgactaatggtctggaaagacaaaacagcctatttccaacttgaaggtcgctatttcacctggtatcagcttgccagctatgcctcgtacatccgtgttcctgtcaactccacggtgtacttggacccctgcatgggccccgccctttgcaacaggagagtcgtcgggtccacccactggggggctgacctcgcggtcaccccttatgattacggcgctaaaattatcctgtctagcgcgtaccatggtgaaatgccccccggatacaaaattctggcgtgcgcggagttctcgttggatgacccagttaagtacaaacatacctgggggtttgaatcggatacagcgtatctgtatgagttcaccggaaacggtgaggactgggaggattacaatgatgcgtttcgtgcgcgccaggaagggaaaatttacaaggccactgccaccagcttgaagttttattttcccccgggccctgtcattgaaccaactttaggcctgaattgaaatgaaatggggtccatgcaaagcctttttgacaaaattggccaactttttgtggatgctttcacggagttcttggtgtccattgttgatatcattatatttttggccattttgtttggcttcaccatcgccggttggctggtggtcttttgcatcagattggtttgctccgcgatactccgtacgcgccctgccattcactctgagcaattacagaagatcttatgaggcctttctttcccagtgccaagtggacattcccacctggggaactaaacatcctttggggttgctttggcaccataaggtgtcaaccctgattgatgaaatggtgtcgcgtcgaatgtaccgcatcatggaaaaatcagggcaggctgcctggaaacaggtggtgagcgaggctacgctgtctcgcattagtagtttggatgtggtggctcattttcagcatctagccgccattgaagccgagacctgtaaatatttggcctcccgactgcccatgctacacaacctgcgcatgacaggttcaaatgtaaccatagtgtataatagcactttgaatcaggtgtttgctatttttccaacccctggttcccggccaaagcttcatgattttcagcaatggttaatagctgtacattcctccatattttcctctgttgcagcttcttgtactctttttgttgtgctgtggttgcgggttccaatactacgtactgtttttggtttccgctggttaggggcaatttttctttcgaactcacagtgaattacacggtgtgtccaccttgcctcacccggcaagcagccacagagatctacgaacccggtaggtctctttggtgcaggatagggtatgaccgatgtgaggaggatgatcatgacgagctagggtttatggtaccgcctggcctctccagcgaaggccacttgactagtgtttacgcctggttggcgttcttgtccttcagctacacggcccagttccatcccgagatattcgggatagggaatgtgagtcgagtttatgttgacatcaaacatcaactcatctgcgccgaacatgacgggcagaacaccaccttgcctcgtcatgacaacatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattaagaccaacaccaccgcagcggcaagctttgctgtcctccaagacatcagttgccttaggcatcgcgactcggcctctgaggcgattcgcaaaatccctcagtgccgtacggcgatagggacacccgtgtatgttactatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaattttaccagctacgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcaattgctttctttgtggtgtatcgtgccgttctgttttgctgtgctcgccaacgccagcaacgacagcagctcccatctacagctgatttacaacttgacgctatgtgagctgaatggcacagattggctagctaacaaatttgattgggcagtggagagttttgtcatctttcccgttttgactcacattgtctcctatggtgccctcactaccagccatttccttgacacagtcgctttagtcactgtgtctaccgccgggtttgttcacgggcggtatgtcctaagtagcatctacgcggtctgtgccctggctgcgttgacttgcttcgtcattaggtttgcaaagaattgcatgtcctggcgctacgcgtgtaccagatataccaactttcttctggacactaagggcagactctatcgttggcggtcgcctgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgcttttggcgttttctattacctacacgccagtgatgatatatgccctaaaggtgagtcgcggccgactgctagggcttctgcaccttttgatcttcctgaattgtgctttcaccttcgggtacatgactttcgcgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttaaaaagcctcgtgttgggtggcagaaaagctgttaaacagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggttagatttaattggcgagaaccatgcggccgaaattaaaaaaaaaaaaaaaaaaaaaaaaaaaaaaaaaaaaaaaaaaaaaaaaaaaaaaaaaaaaaaaacgttgcggccgccctgcagg

(19) United States
(12) Patent Application Publication
(10) Pub. No.: US 2008/0131459 A1
(43) Pub. Date: Jun. 5, 2008

(54) Full-Length Infectious Cdna Clone for Porcine Reproductive and Respiratory Syndrome Virus(Prrsv) and Uses Thereof

(76) Inventors: Seung Han Lee, Kyonggi-do (KR); Young-Min Lee, Chungcheong-buk (KR)

Correspondence Address: JHK LAW, P.O. BOX 1078, LA CANADA, CA, 91012-1078, US

(21) Appl. No.: 11/571,878

(22) PCT Filed: Jul. 9, 2005
(86) PCT No.: PCT/KR05/02220

§ 371 Date: Jan. 9, 2007

(30) Foreign Application Priority Data
Jul. 9, 2004 (KR) 10-2004-0054582

(51) Int. Cl. A61K 39/12; C07H 21/02; C12N 15/00; C12N 5/06; C12Q 1/68; A61K 48/00; C07H 21/04; C12N 7/00

(52) U.S. Cl. 424/204.1; 536/23.1; 435/320.1; 435/243; 435/455; 435/6; 424/93.2; 435/325; 435/235.1

(57) ABSTRACT

The present invention relates to a novel full-length infectious cDNA clone for Porcine Reproductive and Respiratory Syndrome Virus (PRRSV), derivatives therefrom and uses thereof Particularly, the present invention relates to a full-length PRRSV genomic RNA represented by SEQ. ID. No 27 and the genetically stable full-length infectious PRRSV cDNA clone thereof. The PRRSV genomic RNA and infectious PRRSV cDNA clone of the present invention can be used not only for the identification of the PRRSV viral genes, but also for the molecular biological studies including viral replication, transcription and translation. Moreover, it can also be applied to the development of the therapeutic agents, vaccines, diagnostic reagents and diagnostic devices for porcine reproductive and respiratory syndrome and can be used as a novel expression vector for a variety of heterologous genes of interest.

### FIELD OF THE INVENTION

The present invention relates to a full-length genomic RNA of porcine reproductive and respiratory syndrome virus (referred as “PRRSV” hereinafter), infectious PRRSV cDNA clone synthesized from the above RNA, and uses of the cDNA clone and derivatives therefrom, more precisely, a full-length PRRSV genomic RNA, genetically stable infectious PRRSV cDNA clone for the PRRSV genomic RNA represented by SEQ. ID. No 27, clones derived from the mentioned PRRSV cDNA, and uses thereof as a therapeutic agent, a vaccine and a diagnostic agent for PRRSV, and a PRRSV vector for expressing heterologous genes or gene vaccines.

### BACKGROUND OF THE INVENTION

Porcine reproductive and respiratory syndrome (PRRS) was first recognized about a decade ago in North America (Keffaber K. K., American Association Swine Practitioners Newsletter 1:1-9, 1989) and shortly thereafter in Europe (Paton D. J. et al., Vet. Rec. 128: 617, 1991) and Asia (Shimizu et al., J. Vet. Med. Sci. 56: 389-391, 1994). It has since become one of the most common and economically significant infectious diseases in the swine industry worldwide (Albina E., Vet. Microbiol. 55: 309-316, 1997; Dee et al., Vet. Rec. 140: 498-500, 1997). It is characterized by mild to severe reproductive failures in sows and gilts and respiratory problems in piglets (Rossow K. D. Vet Pathol. 35: 1-20, 1998; Done S. H. and Paton D. J, Vet. Rec. 136: 32-35, 1995; Rossow K. D. et al., Vet. Pathol. 32: 361-373, 1995; Christianson W. T. and Joo H. S., Swine Health Production 2: 10-28, 1994). The PRRS virus (PRRSV) was first isolated almost simultaneously in Europe and North America; these strains are designated as Lelystad (Wensvoort G. C. et al., Vet. Q. 13: 121-130, 1991) and VR-2332 (Benfield D. A. et al., J. Vet. Diagn. Invest. 4: 127-133, 1992; Collins J. E. et al., J. Vet. Diagn. Invest. 4: 117-126, 1992), respectively. Although these strains induce phenotypically indistinguishable disease symptoms (Halbur et al., Vet. Pathol. 32: 649-660, 1995), they are genetically (Allende R. T. et al. J. Gen. Virol. 80: 307-315, 1999; Nelsen C. J. et al., J. Virol. 73: 270-280, 1999; Meng X. J. et al., Arch. Virol. 140: 745-755, 1995) and serologically (Wootton S. K. et al., Clin. Diagn. Lab. Immunol. 5: 773-779, 1998; Nelson E. A. et al., J. Clin. Microbiol. 31: 3184-3189, 1993; Wensvoort G. E. et al., J. Vet. Diagn. Invest. 4: 134-138, 1992) distinct. They currently constitute the two distinct genotypes of the known PRRSV strains.

PRRSV belongs to the family Arteriviridae in the order Nidovirales together with equine arteritis virus (EAV), simian hemorrhagic fever virus, and the lactate dehydrogenase-elevating virus of mice (Snijder E. J. and Meulenberg J. J., Fields Virology, 4th ed., 2001; Cavanagh D, Arch. Virol. 142: 629-633, 1997). Like the other arteriviruses, PRRSV is a small-enveloped virus with a positive-sense, single-stranded RNA genome of ≈15 kb in length. The genome has a cap structure at its 5′ end and a poly (A) tail at its 3′ end. The genome contains at least nine open reading frames (ORFs) flanked by 5′ and 3′ noncoding regions (NCRs) (Snijder E. J. and Meulenberg J. J., Fields Virology, 4th ed., 2001; Wu W. H. et al., Virology 287: 183-191, 2001; Conzelmann K. K. et al., Virology 193: 329-339, 1993; Meulenberg J. J. et al. Virology 192: 62-72, 1993). Two overlapping ORFs, ORF1a and 1b, are expressed from the genomic RNA, processed into 13 mature nonstructural proteins, and known to be involved in viral replication (Bautista E. M. et al., Virology 298: 258-270, 2002; Wootton et al., Arch. Virol. 145: 2297-2323, 2000; van Dinten L. C. et al. J. Virol. 73: 2027-2037, 1999). ORFs 2a, 2b, and 3-7 are translated from the 5′ end of a coterminal-nested set of functionally monocistronic subgenomic mRNAs. The small ORF 2b is completely embedded within the larger ORF 2a (Wu W. H. et al., Virology 287: 183-191, 2001). These ORFs are believed to encode the viral structural proteins (Wu W. H. et al., Virology 287: 183-191, 2001; Dea S. C. A. et al., Arch Virol. 145: 659-688, 2000; Meulenberg J. J. et al., Vet. Microbiol. 55: 197-202, 1997; Meulenberg J. J. et al., Virology 192: 62-72, 1993).

To analyze positive-sense RNA viruses such as PRRSV at a molecular and genetic level, a reverse genetics system such as “RNA-launched” or “DNA-launched” is highly desirable, as this system allows us to be able to genetically manipulate the viral genome (Boyer J. C. and Haenni A. L., Virology 198: 415-426, 1994). In the classical “RNA-launched” system, a genetically stable infectious cDNA molecular clone serves as the template for infectious RNA synthesis; cells transfected with these synthetic RNAs then produce the synthetic virus (Casais R. V. et al., J. Virol. 75: 12359-12369, 2001; Yamshchikov V. F. et al., Virology, 281: 294-304, 2001; Almazan F. J. et al., Proc. Natl. Acad. Sci. 97: 5516-5521, 2000; Campbell M. S, and Pletnev A. G. Virology 269: 225-237, 2000; Polo S. G. et al., J. Virol. 71: 5366-5374, 1997; van Dinten L. C. et al., Proc. Natl. Acad. Sci. 94: 991-996, 1997; Gritsun T. S, and Gould E. A., Virology 214: 611-618, 1995; Kapoor M. L. et al., Gene 162: 175-180, 1995; Schlesinger S., Mol. Biotechnol. 3: 155-165, 1995; Rice C. M. et al. New Biol. 1: 285-296, 1989; Ahlquist P. R. et al. Adv. Virus Res. 32: 215-242, 1987; Rice C. M. et al., J. Virol. 61: 3809-3819, 1987; van der Werf S. J. et al., Proc. Natl. Acad. Sci. 83: 2330-2334, 1986; Ahlquist P. and Janda M., Mol. Cell. Biol. 4: 2876-2882, 1984). In the alternative “DNA-launched” system, synthetic viruses are recovered by directly transfecting the cells with infectious cDNA. This approach was first successful for generating polioviruses (Racaniello V. R. and Baltimore D., Science 214: 916-919, 1981), and was later used to produce plant viruses (Boyer J. C. and Haenni A. L. Virology, 198: 415-426, 1994) and alphaviruses (Schlesinger S, and Dubensky T. W., Curr. Opin. Biotechnol. 10: 434-439, 1999).

In both approaches, two requirements must be met. First, the specific infectivity of the synthetic RNAs that are transcribed in vitro from the infectious cDNA must be sufficiently high; this ensures that the system can be used for direct molecular and genetic analyses, such as determining the functions of each viral protein and cis-acting RNA elements in viral replication and pathogenesis (Yun S. I. et al., J. Virol. 77: 6450-6465, 2003). Second, the cloned long viral genome has to remain genetically stable when it is manipulated in a host cell. For many RNA viruses, the genetic instability of the cloned cDNA during its construction is particularly problematic (Yun S. I. et al., J. Virol. 77: 6450-6465, 2003; Casais R. V. et al., J. Virol. 75: 12359-12369, 2001; Thiel V. et al., J. Gen. Virol. 82: 1273-1281, 2001; Yamshchikov, V. et al., Virology 281: 272-280, 2001; Almazan F. et al., Proc. Natl. Acad. Sci. 97: 5516-5521, 2000; Campbell M. S, and Pletnev. A. G. Virology 269: 225-237, 2000; Yount B. and Baric R. S., J. Virol. 74: 10600-10611, 2000; Mendez E. et al., J. Virol. 72: 4737-4745. 1998; Polo S. et al., J. Virol. 71: 5366-5374, 1997; Gritsun T. S, and Gould E. A., Virology 214: 611-618, 1995; Wang C. Y. et al., J. Virol. 68: 3550-3557, 1994; Cunningham T. P. et al., Gene 124: 93-98, 1993; Rice C. M. et al., New Biol. 1: 285-296, 1989). With regard to PRRSV, two infectious cDNAs have been previously constructed to date (Nielsen H. S. et al., J. Virol. 77: 3702-3711, 2003). According to the published literature, however, these cDNAs did not seem to satisfy either of the requirements listed above.

Thus, the present inventors have elucidated a complete full-length nucleotide sequence of virus genomic RNA by using PL97-1/LP1, the first Korean PRRSV isolate, and have developed a reverse genetics system by synthesizing a full-length infectious cDNA for the PRRSV genomic RNA. The present inventors have also completed this invention by confirming that the reverse genetics system using the infectious PRRSV cDNA is effectively used not only for explanation on functions of PRRSV genetic products, self-replication, transcription, translation, and molecular biological mechanisms involved in pathogenicity of PRRSV, but also for the development of a therapeutic agent, a vaccine, a diagnostic agent, and a diagnostic kit for PRRS, in addition to the use as PRRSV vector for the expression of a heterologous gene or a genetic vaccine.

### SUMMARY OF THE INVENTION

It is an object of the present invention to provide novel uses of genomic RNA of PRRSV, infectious PRRSV cDNA, and its derivative cDNA clones as a therapeutic agent, a vaccine and a diagnostic agent for the virus and as a PRRSV vector for the expression of a heterologous gene or a genetic vaccine as well.

In order to achieve the above object,

1) The present invention provides a PRRSV genomic RNA.

2) The present invention provides an infectious PRRSV cDNA, which is able to produce self-replicable infectious PRRSV RNA transcripts.

3) The present invention provides a vector containing cDNA for the above full-length PRRSV genomic RNA.

4) The present invention provides a self-replicable RNA transcript synthesized from the above PRRSV cDNA vector.

5) The present invention provides a recombinant PRRSV obtained from the cells transfected with the RNA transcript synthesized from the PRRSV cDNA vector.

6) The present invention provides a PRRSV expression vector containing the above PRRSV cDNA.

7) The present invention provides various methods for expressing heterologous genes using the PRRSV expression vector above.

8) The present invention provides PRRSV 5′ mutant cDNAs, which lacks 1-15 nucleotides at the 5′ end of the viral genome.

9) The present invention provides PRRSV 5′ mutant cDNAs and their pseudorevertant viruses whose infectivity is recovered by adding various sizes of novel nucleotides to their 5′ ends.

### DESCRIPTION OF DRAWINGS

FIG. 1 is a set of photographs comparing the large-plaque-forming PRRSV isolate PL97-1/LP1 and its original strain PL97-1.

(A) Representative Plaque Morphologies.

Naïve MARC-145 cells were mock-infected or infected with PL97-1 or PL97-1/LP1, overlaid with agarose and the plaques were fixed and stained with crystal violet.

(B) Immunostaining of open reading frame 7(ORF7) of MARC-145 cells infected with PL97-1 or PL97-1/LP1.

Naive MARC-145 cells were mock-infected or infected with PL97-1 or PL97-1/LP1. The infected cells were fixed and stained with an ORF7-specific mouse Mab followed by FITC-conjugated anti-mouse IgG (α-ORF7, green fluorescence) and the results were confirmed by confocal microscopy. The nuclei were visualized by staining with propidium iodide (PI, red fluorescence) in the presence of RNase A. The merged images are also presented.

FIG. 2 is a set of schematic depictions illustrating the assembly of the full-length PRRSV cDNA in bacterial artificial chromosome (BAC) pBeloBAC11.

(A) Schematic depiction of the genomic RNA of the PRRSV PL97-1/LP1 isolate.

Open boxes indicate the PRRSV ORFs. These are flanked by the 5′ and 3′ NCRs, followed by 54 nucleotides of poly (A) tail.

(B) The strategy used to assemble the full-length PRRSV cDNA in the pBeloBAC11 vector.

Six overlapping cDNAs that represent the entire PRRSV PL97-1/LP1 genomic RNA (FrF, Fr1, Fr2, Fr3, Fr4, and FrR) are shown by black thick solid lines. Gray thick solid lines indicate nonviral vector sequences fused immediately upstream of the 5′ end of the viral genome and immediately downstream of the poly (A) tail at its 3′ end. The natural restriction endonuclease recognition sites used for the assembly process are indicated with their positions relative to the complete genome sequence of the PRRSV PL97-1/LP1 isolate (GenBank accession number AY612613). The SP6 polymerase transcription start site (SP6 Promoter) and the three run-off sites (Acl I, Not I, and Sda I) are also indicated.

(C) Schematic depiction of the full-length PRRSV cDNA in the BAC (pBAC/PRRSV/FL).

The complete PRRSV cDNA is under the control of SP6 promoter elements for in vitro transcription. (A)₅₄ indicates the 54 nucleotides of the poly (A) tail.

FIG. 3 is a set of schematic depictions illustrating the full-length PRRSV cDNA templates used for in vitro SP6 polymerase run-off transcription.

(A) Schematic depiction of the full-length PRRSV cDNA (pBAC/PRRSV/FL) that was constructed in the present invention.

The viral ORFs are shown along with black thick solid lines at both termini that represent the 5′ and 3′ NCRs of the viral genome. Gray thick solid lines indicate nonviral vector sequences.

(B) The 5′ and 3′ termini of four SP6-driven full-length PRRSV cDNA templates.

The nucleotide sequences of the PRRSV genomic RNA are shown as bold uppercase letters. The SP6 promoter transcription start and the unique restriction endonuclease recognition site used in run-off transcription are shown at the 5′ and 3′ ends, respectively. Also shown are the 5′ and 3′ termini of the four SP6-driven full-length PRRSV cDNA templates used for SP6 RNA synthesis by in vitro run-off transcription. The 3′-termini of the PRRSV cDNA templates were prepared by complete digestion of pBAC/PRRSV/FL with Sda I (pBAC/PRRSV/FL/SdaI), Not I (pBAC/PRRSV/FL/NotI), or Acl I (pBAC/PRRSV/FL/AclI), resulting in 14 (CGTTGCGGCCGCCC), 10 (CGTTGCGGCC), or 2 (CG) nucleotides of virus-unrelated sequence at their 3′-ends, respectively. In contrast, the authentic 3′ end of PRRSV genomic RNA was presented when pBAC/PRRSV/FL was linearized by digestion with Acl I and then treated with mung bean nuclease (MBN) to remove the virus-unrelated single-stranded dinucleotides CG; this yielded the pBAC/PRRSV/FL/AclI^{MBN} cDNA template. Underlined is the restriction endonuclease recognition site engineered at the 3′ end of the viral genome. An arrowhead indicates a cleavage site.

FIG. 4 is a set of graphs showing the generation of highly infectious RNA transcripts from the full-length PRRSV cDNAs and production of synthetic viruses.

(A) Determination of the specific infectivity of synthetic RNAs transcribed from the full-length PRRSV cDNAs.

Four PRRSV cDNA templates, namely, pBAC/PRRSV/FL/SdaI (Sda I), pBAC/PRRSV/FL/NotI (Not I), pBAC/PRRSV/FL/AclI (Acl I), and pBAC/PRRSV/FL/AclI^{MBN} (Acl I^{MBN}), were used for SP6 polymerase run-off transcription. The specific infectivity of the synthetic RNA transcripts was estimated by infectious center assays, where MARC-145 (□) or BHK-21 (▪) cells were electroporated with 2 μg of the synthetic RNA transcripts, serially 10-fold diluted and plated on monolayers of untransfected MARC-145 cells (3×10⁵) in a 6-well plate. After 6 hr, the cells were overlaid with agarose-containing media and the plaques were visualized by crystal violet staining.

(B) Recovery of synthetic PRRSV viruses.

The culture supernatants of MARC-145 cells transfected with each RNA transcript were collected at 24 hr (□), 48 hr ( [symbol] and 72 hr (▪) post-electroporation and the virus titers were determined. The data shown are from one of two independent experiments, both of which yielded similar results.

FIG. 5 is a set of photographs showing the fact that the full-length PRRSV cDNA template alone is not infectious but is needed for the generation of infectious synthetic RNAs during in vitro transcription.

pBAC/PRRSV/FL/AclI^{MBN} cDNA template was subjected to in vitro SP6 polymerase run-off transcription in the absence (no DNase I) or presence (Ipre-treatment) of DNase I. After synthesis, the transcription reaction was treated with DNase I (DNase I+) or RNase A (RNase A+) for 30 min at 37° C. As a control, the reaction mixture was prepared in the absence of SP6 RNA polymerase (control).

(A) One-twentieth of the reaction mixtures was separated on a 0.6% agarose gel and the cDNA template and RNA transcripts were visualized by staining with ethidium bromide.

(B) The reaction mixtures were then used to transfect BHK-21 cells and the infectious centers of plaques were estimated.

FIG. 6 is a set of photographs showing the comparison of synthetic PRRSV viruses recovered from the four infectious PRRSV cDNA templates with the parental virus PL97-1/LP1. (A-B) Naive MARC-145 cells were mock-infected (plate 1) or infected with the parent (plate 2) or synthetic viruses (plates 3-6).

(A) Representative plaque morphology.

A monolayer of infected cells was overlaid with agarose and stained with crystal violet.

(B) Immunofluorescence analysis of ORF7 protein expression.

Infected cells were fixed and stained with an ORF7-specific mouse Mab (green fluorescence) or propidium iodide (red fluorescence) in the presence of RNase A, followed by confocal microscopy.

(C) Growth kinetics in MARC-145 cells of synthetic PRRSV viruses and the parent PL97-1/LP1 at an MOI of 1.

Viruses were harvested at the indicated hours post-infection (h.p.i) and their titers were determined by plaque assays. The data are from one of two independent experiments, both of which yielded similar results.

, PRRSV/FL/SdaI; ▴, PRRSV/FL/NotI; ▪, PRRSV/FL/AclI; □, PRRSV/FL/AclI^{MBN}; Δ, PL97-1/LP1 viruses.

FIG. 7 is a set of photographs showing that recombinant PRRSV retains a Cla I genetic marker engineered into the infectious PRRSV cDNA.

(A) Schematic depiction of the RT-PCR fragments of PRRSV/FL/AclI^{MBN} and PRRSV/FLgm/AclI^{MBN} expected after Cla I digestion.

The primers used for RT-PCR (arrows) and the engineered Cla I site (asterisk) are shown. The 2101-bp RT-PCR fragment amplified from genomic RNA of the parental virus should not be cleaved by Cla I while the fragment from the recombinant virus should be cleaved into 1199-bp and 902-bp products.

(B) Agarose gel analysis of the genetic marker.

Naive BHK-21 cells were electroporated with synthetic RNAs transcribed from the pBAC/PRRSV/FL/AclI^{MBN} or pBAC/PRRSV/FLgm/AclI^{MBN} cDNA. Viruses harvested 72 hr later were serially passaged in MARC-145 cells at an MOI of 0.1. At each passage, the viruses were treated with DNase I and RNase A for 30 min at 37° C. prior to the next round of infection (Yun et al., J. Virol. 77: 6450-6465, 2003; Mendez et al., J. Virol. 72: 4737-4745, 1998). At passages 1 and 3, viral RNA from the released viruses was subjected to RT-PCR. The resulting products were treated with (+) or without (−) Cla I, separated on a 1% agarose gel and visualized by ethidium bromide staining. The expected sizes of the undigested and digested PCR products are shown on the right. M, 1-kb DNA marker.

FIG. 8 is a graph showing that highly infectious RNA transcripts are synthesized from infectious PRRSV cDNAs passaged for 240 generations in E. coli.

Two independent clones carrying pBAC/PRRSV/FL ( and ∘) were propagated in 10 ml of 2xYT in the presence of 12.5 μg/ml chloramphenicol at 37° C. for 12 days by daily 10⁶-fold dilutions with fresh broth. At the indicated passages, the DNA plasmids were purified, linearized by Acl I digestion and MBN treatment, and used as templates for run-off transcription. The specific infectivity of the RNA transcripts from BHK-21 cells was then determined.

FIG. 9 is a set of graphs showing that the cap structure and poly (A) tail of the viral genome are required for viral replication.

(A) Requirement of the cap structure.

Run-off transcription from pBAC/PRRSV/FL/AclI^{MBN} cDNA template was performed in the presence or absence (None) of the m⁷G(5′)ppp(5′)A or m⁷G(5′)ppp(5′)G cap analog. BHK-21 cells were electroporated with the synthetic RNAs and infectious plaque centers were visualized.

(B) Schematic depiction of the pBAC/PRRSV/FLnop(A)/XbaI^{MBN} construct that was used to synthesize unpolyadenylated RNA transcripts.

pBAC/PRRSV/FLnop(A) was generated by introducing a unique Xba I site immediately upstream of the poly (A) tail of pBAC/PRRSV/FL that served as a run-off site. Run-off transcription of this Xba I-linearized MBN-treated cDNA template produced capped RNA transcripts terminating with -GCC GAA ATT¹⁵⁴¹¹ and lacking the poly (A) 54 nucleotides. The restriction endonuclease recognition sites used for run-off transcription (Acl I for pBAC/PRRSV/FL/AclI^{MBN} and Xba I for pBAC/PRRSV/FLnop(A)/XbaI^{MBN}) are underlined. An arrowhead indicates the cleavage site.

(C) Requirement of the poly (A) tail.

Determination of the specific infectivity of synthetic RNAs transcribed from pBAC/PRRSV/FL/AclI^{MBN} and pBAC/PRRSV/FLnop(A)/XbaI^{MBN} cDNAs by run-off transcription in the presence of the m⁷G(5′)ppp(5′)A cap analog. BHK-21 cells were electroporated with the synthetic RNAs and infectious plaque centers were visualized.

FIG. 10 is a schematic depiction of luciferase (LUC)-expressing PRRSV viral replicons.

The three viral replicons pBAC/PRRSV/RepLuc MB, pBAC/PRRSV/RepLuc ME, and pBAC/PRRSV/RepLuc DI have large internal deletions of nt 12714-14194, nt 12163-14194, and nt 12163-15252, respectively. The other set of eight viral replicons have a large internal deletion from nt 12163 to nt 15200, 15150, 15100, 15050, 15000, 14950, 14900, and 14500 for pBAC/PRRSV/RepLuc S1-S8, respectively. An expression cassette consisting of the EMCV IRES-driven LUC gene was inserted at the deletion site to facilitate the monitoring of viral replication.

FIG. 11 is a graph showing the induction of LUC gene expression in the PRRSV viral replicons of FIG. 10.

Naive BHK-21 cells (8×10⁶) were transfected with 2 μg of the replicon RNAs transcribed from each cDNA template and seeded on 6-well plates at a density of 4×10⁵ cells per well. At the indicated time points, lysates were prepared for LUC assays. The experiments were performed in triplicate; mean values are shown.

▪, pBAC/PRRSV/RepLuc MB; ▴, pBAC/PRRSV/RepLuc ME;

♦, pBAC/PRRSV/RepLuc DI; Δ, pBAC/PRRSV/RepLuc S1;

*, pBAC/PRRSV/RepLuc S2; ∘, pBAC/PRRSV/RepLuc S3;

X, pBAC/PRRSV/RepLuc S4; [symbol] pBAC/PRRSV/RepLuc S5;

□, pBAC/PRRSV/RepLuc S6; ⋄, pBAC/PRRSV/RepLuc S7;

, pBAC/PRRSV/RepLuc S8.

FIG. 12 is a set of photographs showing that the 911 nucleotides at the 3′ end of the viral genome contain a cis-acting element that is required for replication.

(A) Schematic depiction of the ORF7 protein expression cassette that is based on the Sindbis virus-derived expression vector pSinRep19, which is a double subgenomic noncytopathic RNA vector.

The pSinRep19/PRRSV ORF7 vector encodes the ORF7 and PAC genes, which are expressed by separate subgenomic promoters (26S promoter) as indicated by arrows. MCS, multiple cloning sites.

(B) Generation of a BHK-21 cell line that stably expresses ORF7 protein.

This cell line was established by transfection with in vitro transcribed SinRep19/PRRSV ORF7 RNAs followed by selection with puromycin. A SinRep19 vector RNA-expressing control cell line was also established. The cells were fixed and stained with an ORF7-specific mouse Mab (green fluorescence) and propidium iodide (red fluorescence) in the presence of RNase A, followed by confocal microscopy. Merged images were also presented.

(C) Assessment of the replicability of the PRRSV viral replicons when ORF7 protein was provided in trans.

ORF7-expressing BHK-21 cells were transfected with PRRSV/RepLuc S6 (∘), PRRSV/RepLuc S7 (□), or PRRSV/RepLuc S8 (Δ) RNAs. SinRep19 RNA-selected BHK-21 cells were also transfected with PRRSV/RepLuc S6 (X), PRRSV/RepLuc S7 ( [symbol] ), or PRRSV/RepLuc S8 (*) RNAs. Naive BHK-21 cells were also transfected with PRRSV/RepLuc S6 (), PRRSV/RepLuc S7 (▪), or PRRSV/RepLuc S8 (▴) RNAs. LUC activity in the transfected cells was then determined at the indicated time points. The experiments were performed in triplicate; mean values are shown.

(D) Assessment of the replicability of the PRRSV viral replicons when co-transfected with infectious PRRSV/FL/AclI^{MBN} RNA transcripts.

BHK-21 cells were co-transfected with PRRSV/FL/AclI^{MBN} RNA and PRRSV/RepLuc S6 (∘), PRRSV/RepLuc S7 (□), or PRRSV/RepLuc S8 (Δ) RNA. Naive BHK-21 cells were also transfected with only PRRSV/RepLuc S6 (), PRRSV/RepLuc S7 (▪), or PRRSV/RepLuc S8 (▴) RNAs. LUC assays were then performed as described above. These experiments were performed in triplicate; mean values are shown.

FIG. 13 is a set of photographs showing the expression of the EGFP reporter gene by infectious PRRSV cDNA/recombinant viruses.

(A) Schematic depiction of the two recombinant infectious PRRSV cDNAs pBAC/PRRSV/FL/IRES-EGFP and pBAC/PRRSV/FL/N^{pro}-EGFP and the parental cDNA.

The depiction of pBAC/PRRSV/FL/IRES-EGFP indicates the EMCV IRES-driven EGFP expression unit that is fused to the first 33 nucleotides (nt 14921) of the ORF7 coding region and that is followed by the nucleotide sequence of the viral genome from nt 14501 to the end, including the poly (A) tail. The depiction of pBAC/PRRSV/FL/N^{pro}-EGFP shows that the autoprotease N^{pro} gene of bovine viral diarrhea virus (N^{pro}) is fused adjacent to the N-terminus of EGFP so that the correct N-terminus of the EGFP protein is created by cleavage with N^{pro}. N^{pro}-EGFP was then fused to the first 33 nucleotides (nt 14921) of the ORF7 coding region that include a short PRRSV transcription-regulating sequence that is required for the synthesis of the subgenomic mRNA of the N^{pro}-EGFP gene. N^{pro}-EGFP is then followed by the nucleotide sequence from nt 14501 to the end, including the stretch of poly (A) tail.

(B) Immunofluorescence analysis assessing the expression of EGFP and the ORF7 protein.

Naive BHK-21 cells were transfected with 2 μg of synthetic RNAs transcribed from pBAC/PRRSV/FL (plates 1-6), pBAC/PRRSV/FL/IRES-EGFP (plates 7-12), or pBAC/PRRSV/FL/N^{pro}-EGFP (plates 13-18) and 36 hr later were fixed and stained with an ORF7-specific mouse Mab followed by Cy3-conjugated anti-mouse IgG (α-ORF7, red fluorescence) and confocal microscopy. EGFP proteins were recognized under an appropriate filter as green fluorescence. Nuclei were visualized by staining with 4′,6-diamidino-2-phenylindole (DAPI, blue fluorescence) The merged images are also presented.

(C) Production of recombinant EGFP-expressing PRRSVs.

Naive MARC-145 cells were transfected with 2 μg of synthetic RNAs transcribed from pBAC/PRRSV/FL, pBAC/PRRSV/FL/IRES-EGFP, or pBAC/PRRSV/FL/N^{pro}-EGFP and the virus titers in their culture supernatants were determined 24 hr (□), 48 hr ( [symbol] ), and 72 hr (▪) post-transfection by measuring the green focus-forming units per ml (GFU/ml) for the EGFP-expressing viruses or the PFU/ml for pBAC/PRRSV/FL.

FIG. 14 is a schematic diagram of PRRSV 5′ end serial deletion mutations introduced at the utmost 5′ end of the viral genome. PRRSV-specific sequences are shown in boldface and uppercase type. Hyphens indicate the deleted nucleotide sequences.

FIG. 15 presents the importance of PRRSV 5′ end nucleotide sequences for replication and recovery of the adapted pseudorevertants.

Specific infectivities of the synthetic RNA transcripts were derived from recombinant cDNAs and representative plaque/focus morphology. For focus/plaque morphology, the cells were immunostained with an anti-PRRSV ORF7 mouse Mab and a peroxidase-conjugated goat anti-mouse IgG, and stained with DAB substrate (for foci), and subsequently the same cells were stained with crystal violet (for plaques).

FIG. 16 presents the isolation and characteristics of adapted pseudorevertants.

The culture supernatants from the electroporated cells were passaged on naïve MARC-145 cells to recover pseudorevertants. Upon infection of naïve MARC-145 cells with equal amounts of the pseudorevertants at the indicated time points, level of PRRSV Nsp1a viral protein expression was examined by immunoblotting with an anti-PRRSV Nsp1a rabbit antiserum. In parallel, GAPDH protein was detected as a loading and transfer control with an anti-GAPDH rabbit antiserum.

FIG. 17 is a graph showing growth kinetics of adapted pseudorevertant viruses in MARC-145 cells at an MOI of 0.01.

▪: PRRSV/FL; ∘: PRRSV/FL/Δnt1; Δ: PRRSV/FL/Δnt3;

□: PRRSV/FL/Δnt5; ⋄: PRRSV/FL/Δnt7.

FIG. 18 shows representative foci/plaques of adapted pseudorevertant viruses. The same plates were stained for foci and plaques as described above.

FIG. 19 presents discovery of novel PRRSV 5′ sequences acquired in adapted pseudorevertants.

Sequence determination of the 5′ end region of pseudorevertants by 5′ RACE. Indicated are a total number of independently picked clones containing the insert the present inventors sequenced and a number of clones containing the particular sequence the present inventors discovered. Novel 5′ sequences are indicated by boldface type. Hyphens indicated the deleted nucleotide sequences in the parental construct.

FIG. 20 shows specific infectivities of the reconstructed PRRSV cDNAs containing the novel PRRSV 5′ sequences discovered by the present inventors and their representative foci/plaques. The same dishes were stained for foci and plaques.

### DETAILED DESCRIPTION OF THE INVENTION

I. The present invention provides a PRRSV genomic RNA.

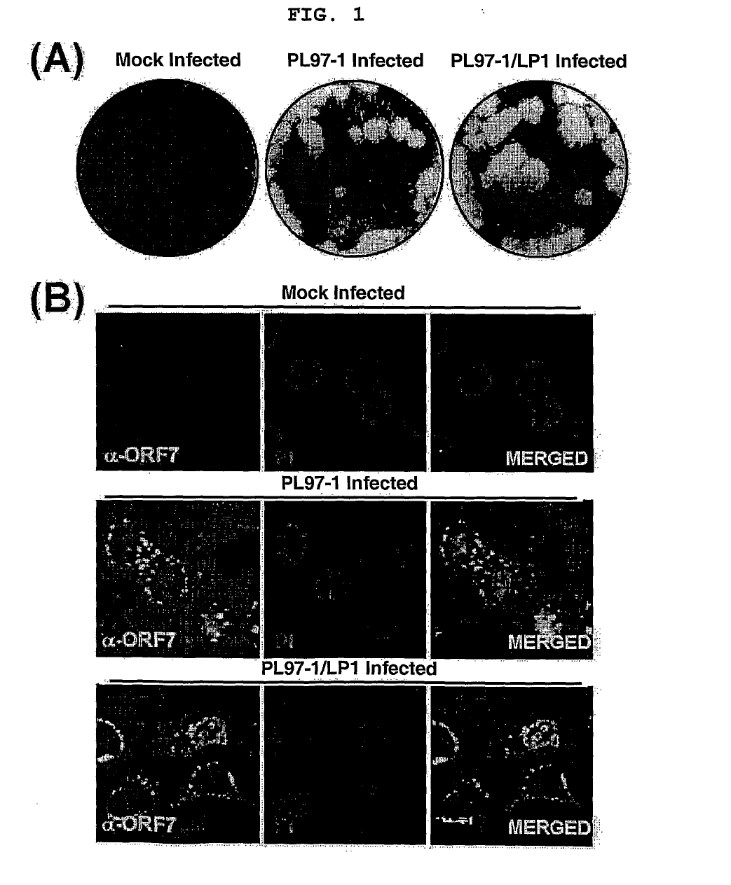

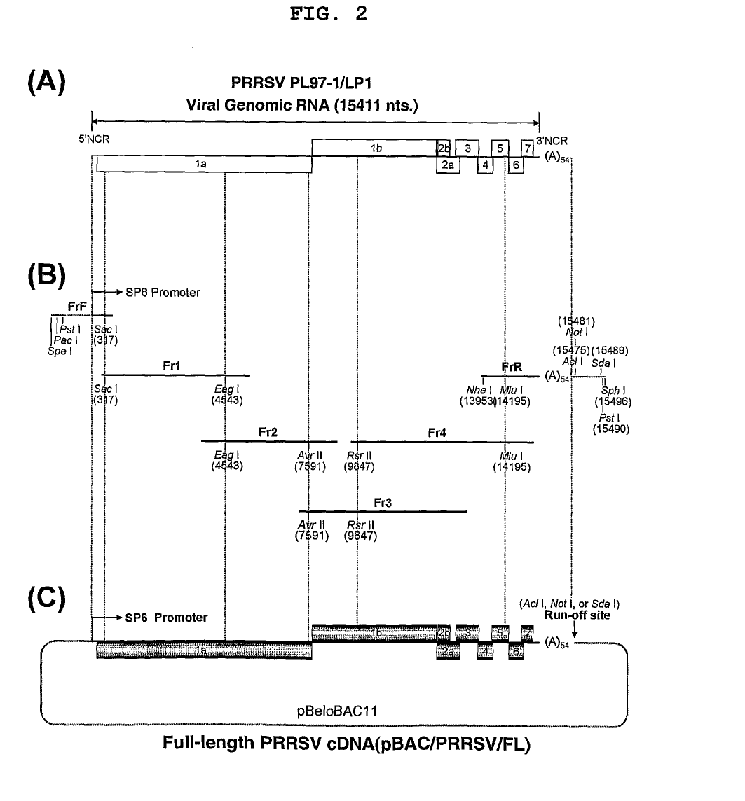

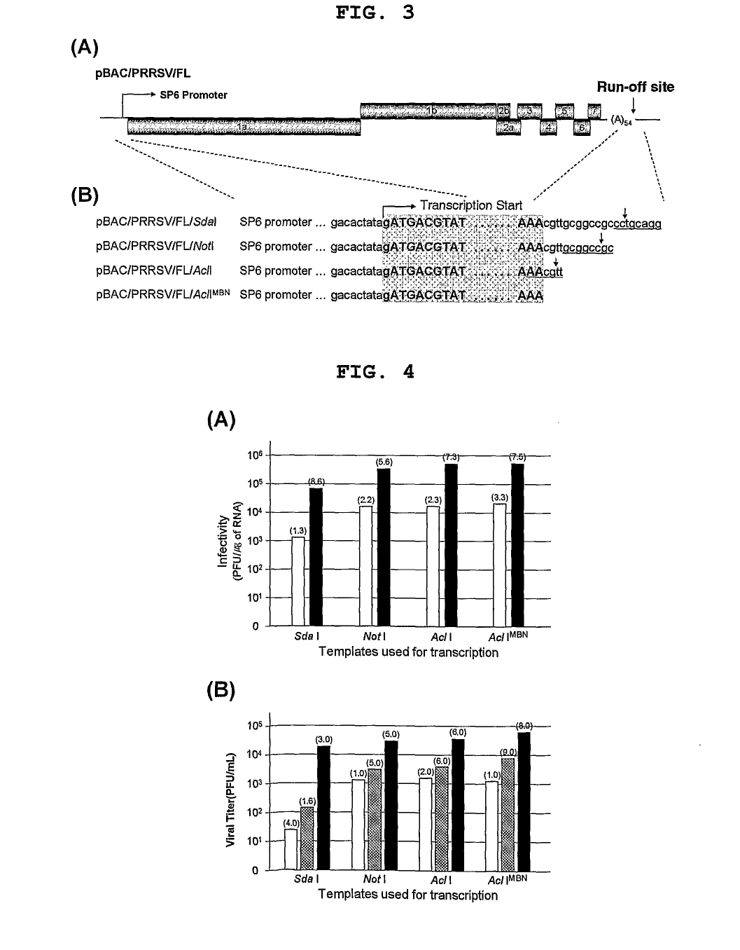

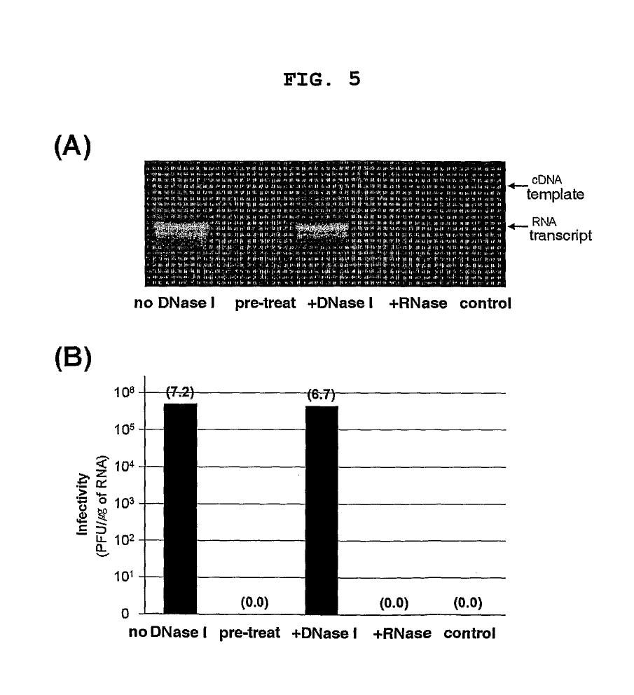

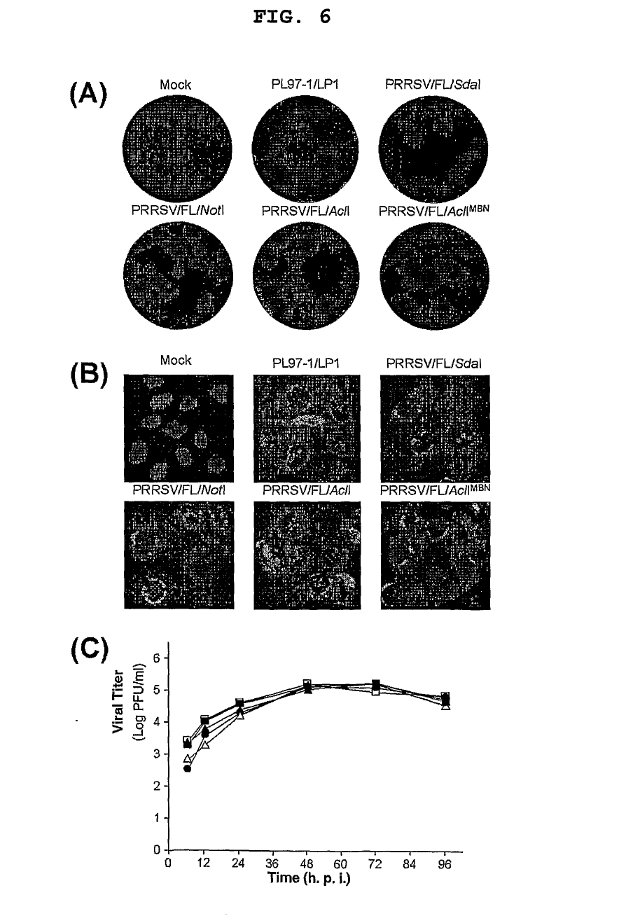

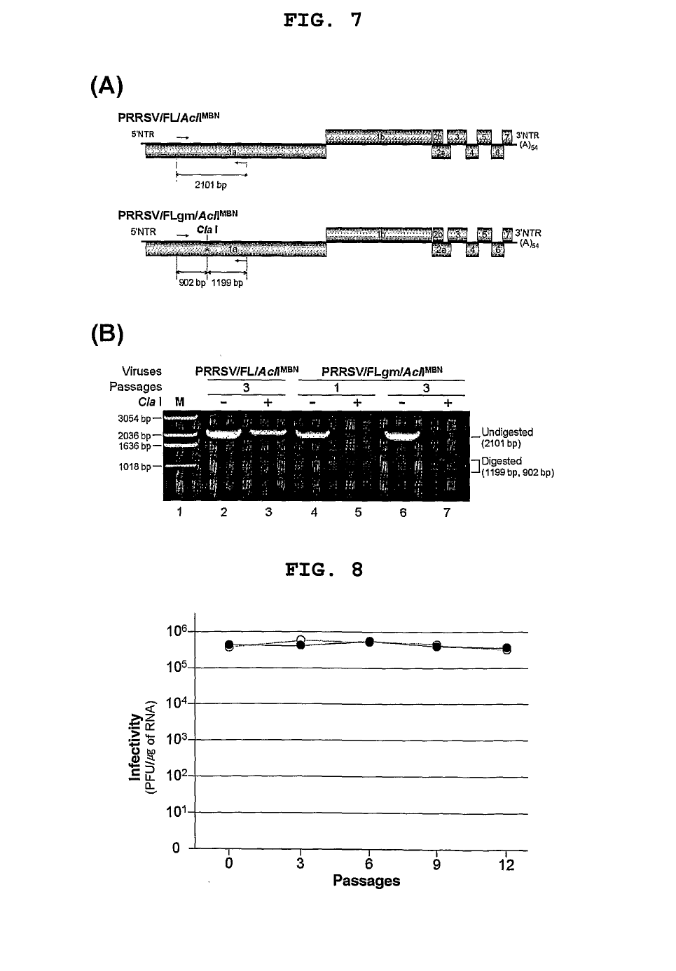

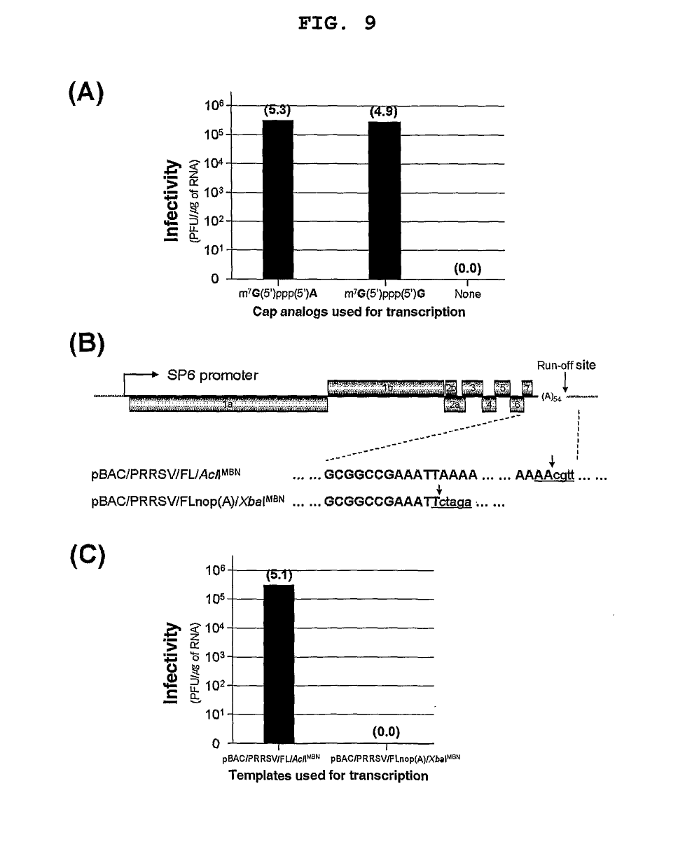

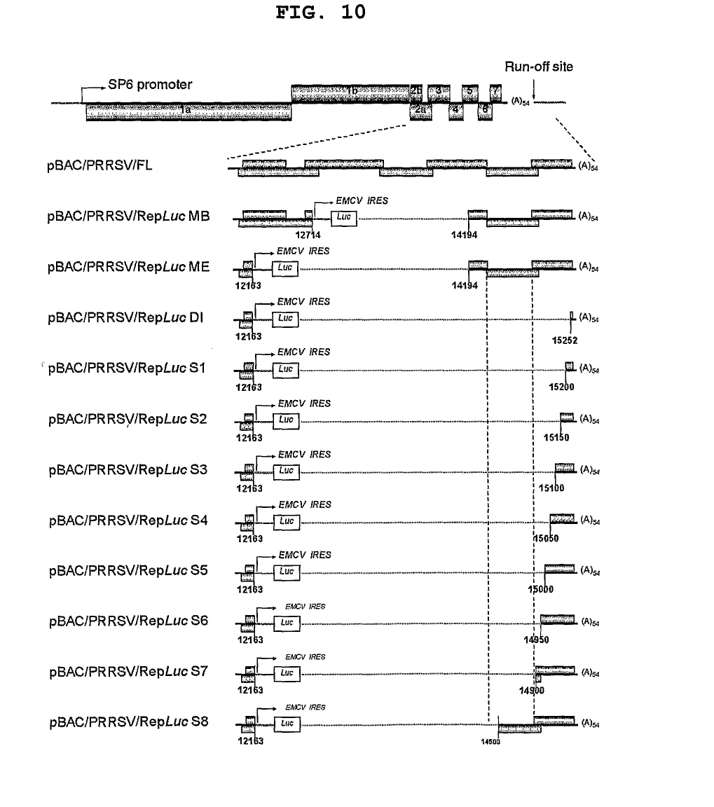

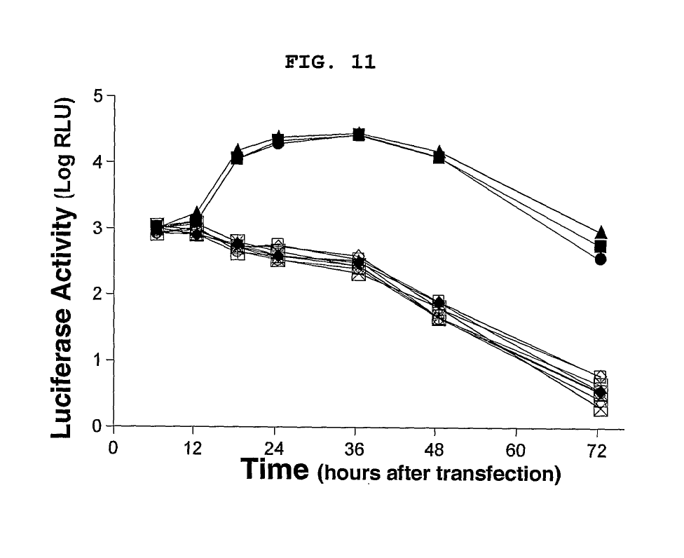

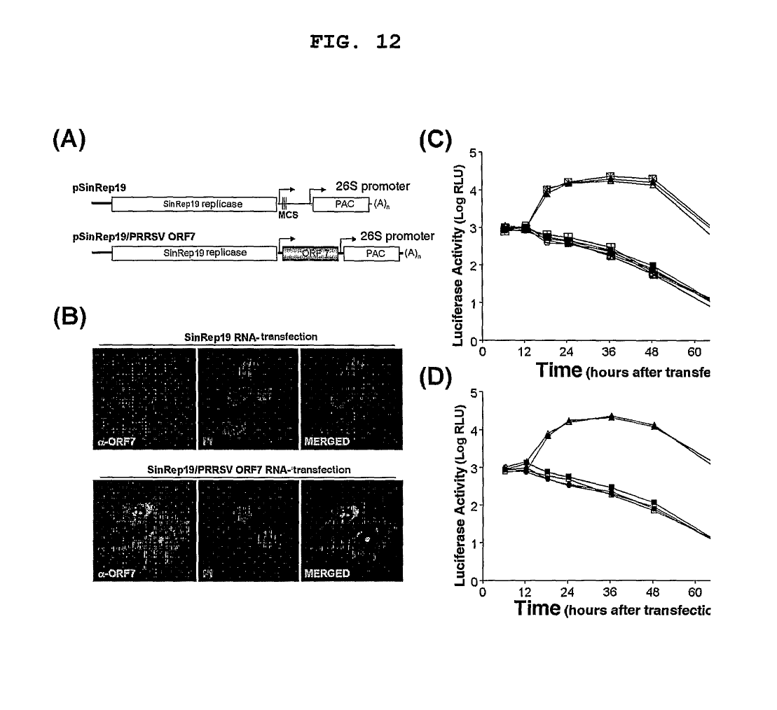

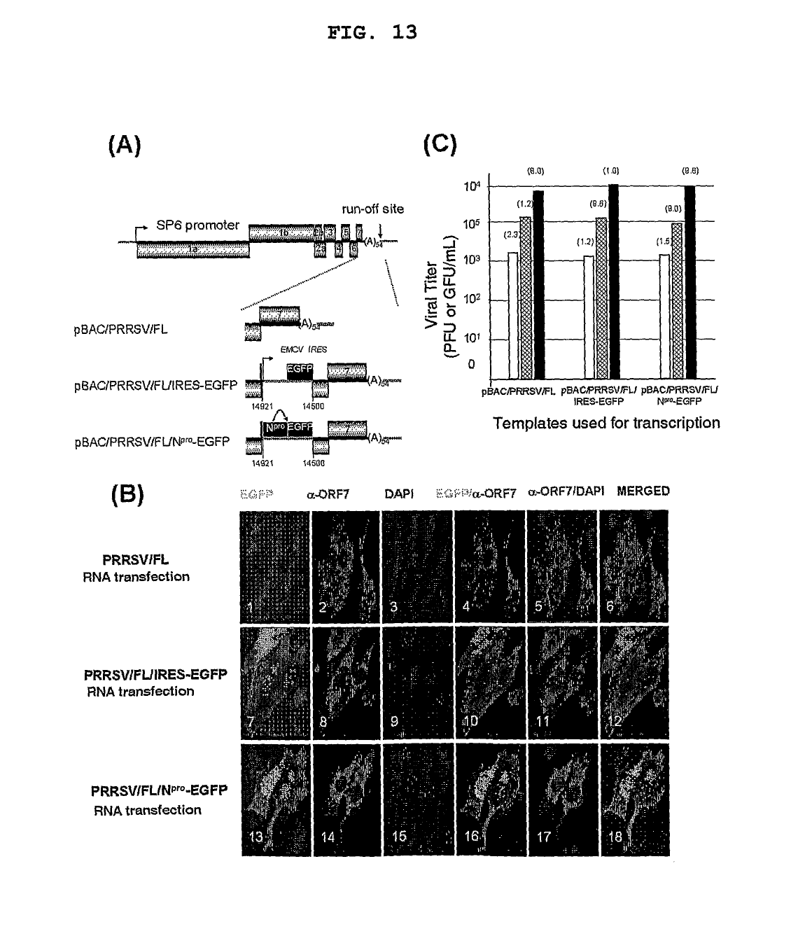

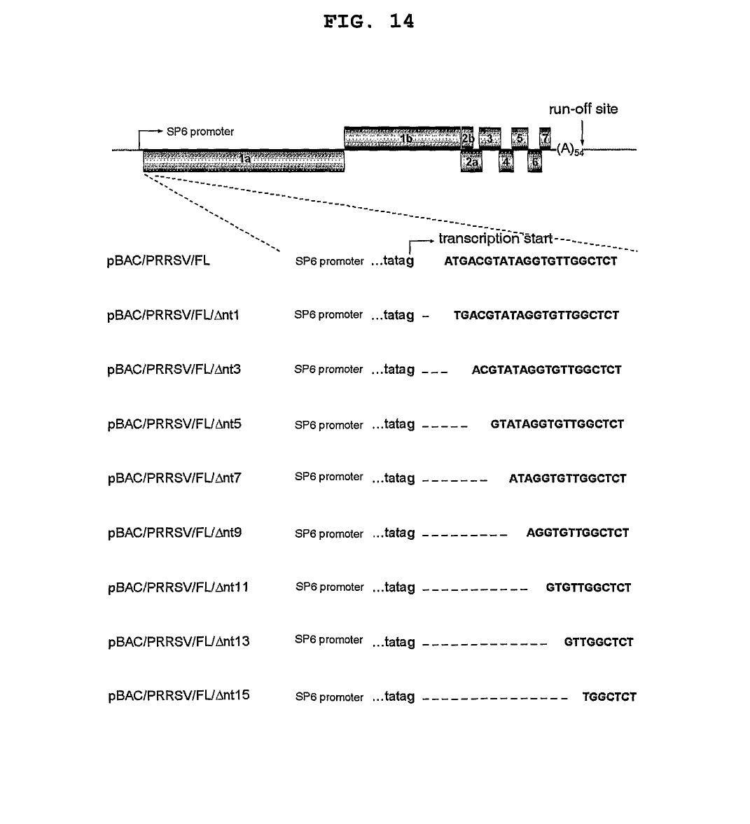

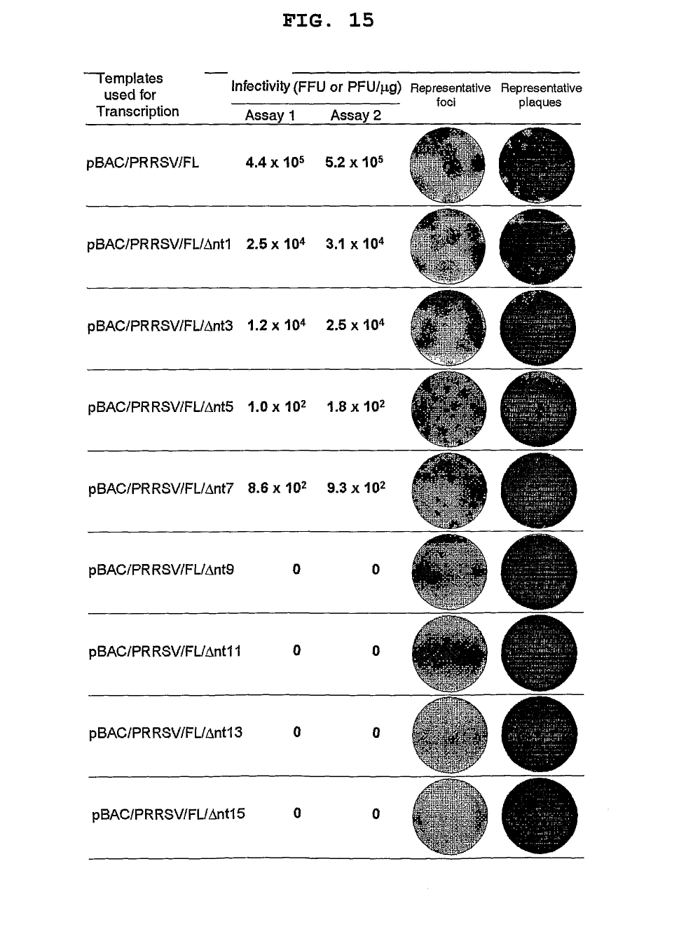

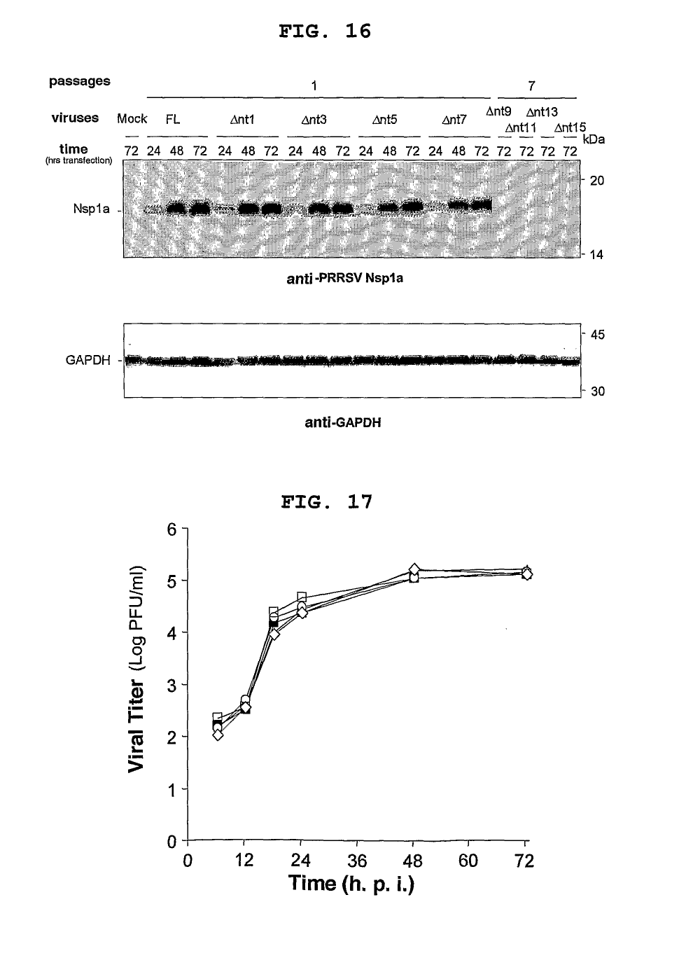

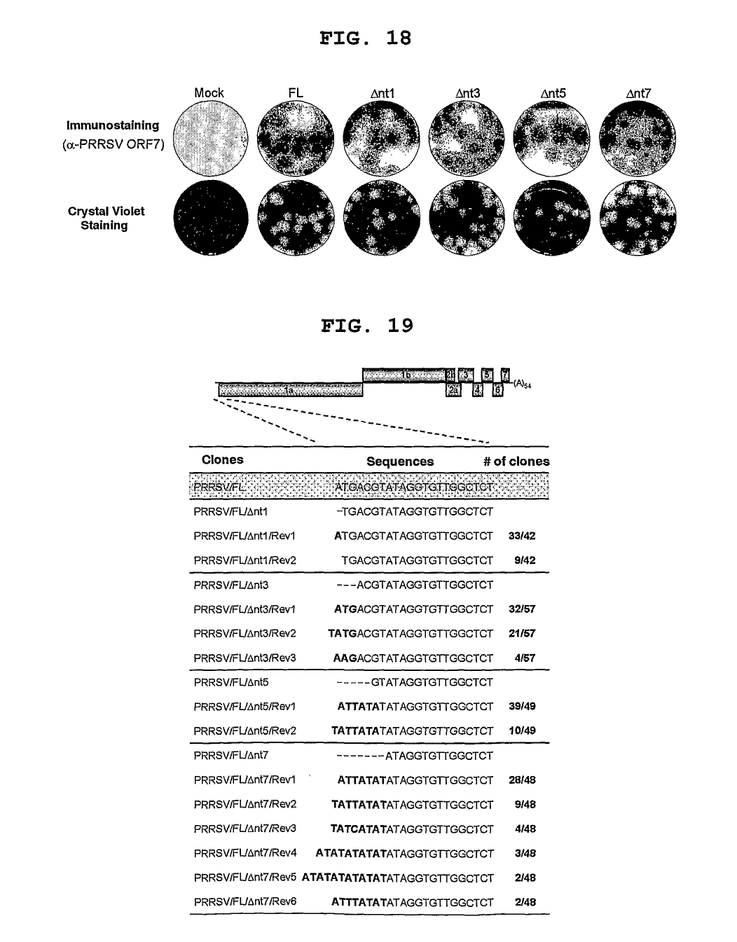

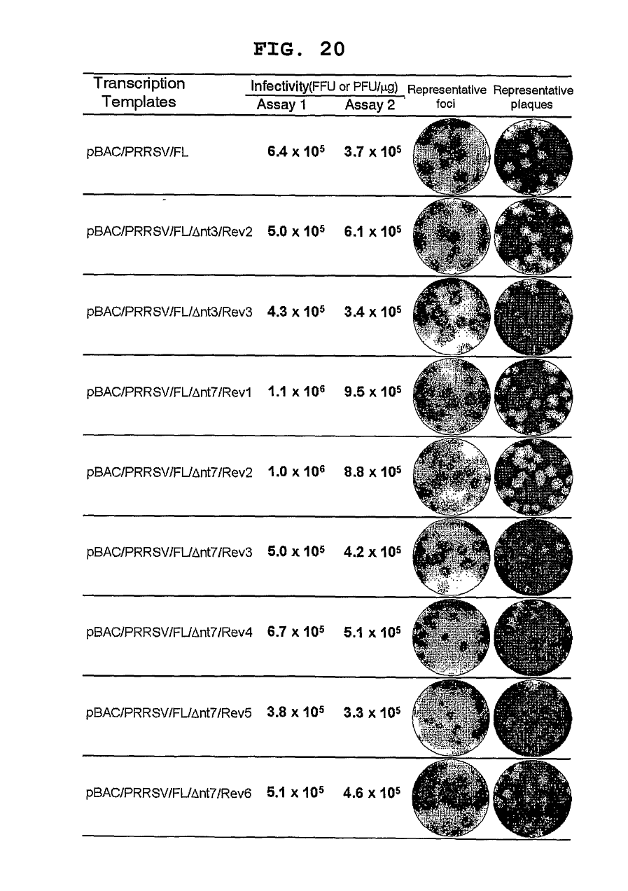

A Korean PRRSV isolate used in the present invention, was named “PRRSV PL97-1/LP1” and prepared as follows; MARC-145 cells were infected with PRRSV PL97-1, the first Korean PRRSV isolate isolated from the PRRSV-infected porcine serum in 1997. A homogeneous population of large plaques (LP) was isolated by plaque-purification technique therefrom, resulting in PRRSV PL97-1/LP1 (see FIG. 1).

In order to elucidate the full-length nucleotide sequence of PRRSV PL97-1/LP1, RT-PCR was performed to synthesize and amplify the four overlapping cDNAs, that is Fr1 (nt 180-5297), Fr2 (nt 3708-9108), Fr3 (nt 7570-13051) and Fr4 (nt 9610-15238) cDNAs, for the full-length sequence of the virus except for the 5′ and 3′ ends, resulting in the four cDNA fragments, which are approximately 5.1 kbp (Fr1), 5.4 kbp (Fr2), 5.5 kbp (Fr3) and 5.6 kbp (Fr4) (see FIGS. 2A and 2B).

5′RACE method was slightly modified to investigate the nucleotide sequence of the 5′ end of PRRSV PL97-1/LP1 genomic RNA. The first-strand cDNA was synthesized from the virus genomic RNA, and then the RNA in the first-strand cDNA-RNA hybrid was degraded. The remaining first-strand cDNA was purified by phenol extraction. In order to insert a primer-binding site, the 3′ end of the first-strand cDNA was ligated with the synthetic oligonucleotide PRX represented by SEQ. ID. No 14. Then, the cDNA was amplified by PCR. The 334-bp PstI-SacI fragment of the cDNA amplicon (FrF) was cloned into the pRS2 vector digested with PstI-SacI, resulting in the construction of pRS/PRRSV/FrF.

3′RACE method (Yun et al., J. Virol. 77: 6450-6465, 2003) was also used to identify the nucleotide sequence of the 3′ end of PRRSV PL97-1/LP1 genomic RNA. At that time, PRX oligonucleotide represented by SEQ. ID. No 14 was ligated to the 3′ end of the viral genomic RNA to prepare a specific primer binding site for RT-PCR, and cDNA was synthesized from the complex using Superscript II RT and PRXR primer, which was then amplified by PCR. The 1537-bp NheI-PstI, fragment of the cDNA amplicon (FrR) was cloned into the pGEM3Z vector digested with XbaI and PstI, resulting in the construction of pGEM/PRRSV/FrR.

Nucleotide sequences of pRS/PRRSV/FrF and pGEM/PRRSV/FrR were analyzed. As a result, the present inventors have elucidated a complete full-length nucleotide sequence of PRRSV PL97-1/LP1 represented by SEQ. ID. No 18. The full-length PRRSV PL97-1/LP1 genomic RNA was composed of 15,411 nucleotides, behind which poly (A) tails of 54 nucleotides were placed. 15,411 nucleotides of the virus genomic RNA were composed of three regions of 5′-noncoding region, virus protein coding region, and 3′-noncoding region.

II. The present invention provides an infectious PRRSV cDNA, which is able to produce self-replicable infectious PRRSV RNA transcripts.

The infectious PRRSV cDNA of the present invention is synthesized based on the nucleotide sequence represented by SEQ. ID. No 18, and is also used as a template for the synthesis of self-replicable infectious PRRSV RNA transcripts through in vitro transcription. The full-length PRRSV cDNA of the present invention, which is represented by SEQ. ID. No 68, can be prepared by amplifying the virus genomic RNA harboring the authentic 5′ and 3′ ends by RT-PCR to produce several overlapping cDNAs, and then assembling them.

To enable in vitro run-off transcription, the SP6 RNA polymerase promoter sequence was placed precisely at the beginning of the viral sequence. To generate an artificial run-off site, a unique restriction endonuclease recognition site was placed immediately downstream of the viral sequence.

In the preferred embodiment of the present invention, SP6-driven full-length PRRSV cDNA was prepared by using four overlapping cDNAs (Fr1, Fr2, Fr3 and Fr4) corresponding to PRRSV genomic RNA, the 5′ end region containing SP6 promoter sequence, and the 3′ end region containing AclI, NotI, SdaI recognition sites in a row, which is used as a run-off site (see FIGS. 2B and 2C).

However, it is common knowledge for the people in this field to use other promoters in addition to the above-mentioned promoter. The full-length PRRSV cDNA provided by the present invention uses AclI, NotI and SdaI as a run-off site but other restriction enzymes can take the place of them.

III. The present invention provides a vector containing cDNA for the above full-length PRRSV genomic RNA.

The vector of the present invention includes full-length infectious PRRSV cDNA. Difficulty of previous efforts to synthesize full-length infectious PRRSV cDNA was genetic instability of cloned PRRSV cDNA (Nielsen et al., J. Virol. 77: 3702-3711, 2003; Meulenberg et al., J. Virol. 72: 380-387, 1998). The cDNA template was used to synthesize infectious RNA transcripts in vitro, but the specific infectivity of the transcripts was approximately 400-1500 cells per 1 μg of RNA (Meulenberg et al., J. Virol. 72: 380-387, 1998). Therefore, reverse genetics system using the conventional infectious PRRSV cDNA is not very efficient for direct molecular biological or genetic analysis of PRRSV.

The present inventors have made efforts to overcome the genetic instability of the cloned PRRSV cDNA by cloning it into a bacterial artificial chromosome (BAC), found in E. coli constantly by one or two copies. The genetic structure of the resultant full-length infectious PRRSV cDNA BAC and the functional integrity of the cloned PRRSV cDNA were confirmed to be maintained stably at least for 240 generations in E. coli (see FIG. 8). In conclusion, the present inventors have overcome the genetic instability of the full-length PRRSV cDNA by using BAC as a vector vehicle, and thus paved the way to treat synthesized PRRSV cDNA stably.

In a preferred embodiment of the present invention, infectious PRRSV cDNA pBAC/PRRSV/FL vector represented by SEQ. ID. No 27 which contains a SP6 promoter is provided (see FIG. 2 and FIG. 3). The present inventors deposited E. coli DH10B (DH10B/pBAC/PRRSV/FL) transformed with the pBAC/PRRSV/FL vector at Korean Collection for Type Cultures (KCTC) of Korea Research Institute of Bioscience and Biotechnology (KRIBB) on Jun. 15, 2004 (Accession No: KCTC 10664BP).

IV. The present invention provides a self-replicable RNA transcript synthesized from the above PRRSV cDNA vector.

In the case of in vitro run-off transcription reaction, the PRRSV cDNA used as a template can be linearized by the digestion with AclI, NotI or SdaI restriction endonuclease. The three linearized plasmids (pBAC/PRRSV/FL/AclI, pBAC/PRRSV/FL/NotI and pBAC/PRRSV/FL/SdaI) are used as a template for the synthesis of RNA transcripts through SP6 polymerase run-off transcription reaction in the presence of m⁷G(5′)ppp(5′)A cap structure analog. The 3′ end of the RNA transcripts synthesized from the cDNA templates of pBAC/PRRSV/FL/AclI, pBAC/PRRSV/FL/NotI or pBAC/PRRSV/FL/SdaI harbors 2 (CG), 10 (CGTTGCGGCC), or 14 (CGTTGCGGCCGCCC) nucleotides of virus-unrelated sequences, respectively. These virus-unrelated nucleotides placed at the 3′ end of synthesized RNA transcripts can be eliminated by using pBAC/PRRSV/FL/AclI^{MBN} as a template for in vitro run-off transcription reaction (see FIG. 3). To prepare the linearized pBAC/PRRSV/FL/AclI^{MBN}, pBAC/PRRSV/FL cDNA was digested with AclI, and then treated with mung bean nuclease (MBN).

To quantify specific infectivity of the PRRSV RNA transcripts synthesized from the above PRRSV cDNA templates, the present inventors performed infectious center assays. As a result, significant specific infectivities (5.6-7.5×10⁵ PFU/μg) were estimated in BHK-21 cells transfected with the RNA transcripts synthesized from pBAC/PRRSV/FL/AclI^{MBN}, pBAC/PRRSV/FL/AclI, and pBAC/PRRSV/FL/NotI as a template (see FIG. 4A). And also, 1.0-2.0×10³ PFU/ml of virus was generated in MARC-145 cells 24 hours after the transfection with the RNA transcripts synthesized from pBAC/PRRSV/FL/AclI^{MBN}, pBAC/PRRSV/FL/AclI or pBAC/PRRSV/FL/NotI as a template. 48 hours after the transfection, cytopathic effect (CPE) was clearly detected, and virus titer was increased up to 5.0-9.0×10³ PFU/ml, approximately 5-10 times higher than that of 24 hours after the transfection. 72 hours after the transfection, strong cytopathic effect was observed in almost every cells and the virus titer went up to 5.0-8.0×10⁴ PFU/ml (see FIG. 4B). From the above results, infectious PPRSV RNA transcripts were generated from the full-length PRRSV cDNA.

When BHK-21 cells were transfected with the RNA transcripts synthesized from pBAC/PRRSV/FL/SdaI as a template, the specific infectivity was 8.6×10⁴ PFU/μg (see FIG. 4A). Thus comparison of specific infectivities of the RNA transcripts (5.6-7.5×10⁵ PFU/μg) synthesized from pBAC/PRRSV/FL/AclI^{MBN}, pBAC/PRRSV/FL/AclI or pBAC/PRRSV/FL/NotI as a template revealed that specific infectivity of these synthetic RNA transcripts was not affected by 2-10 nucleotides of virus-unrelated sequences placed at the 3′ end of the RNA transcripts, but the specific infectivity was decreased by the presence of 14 nucleotides of virus-unrelated sequences at the 3′ end of the RNA transcripts. The reduced infectivity of the RNA transcripts was also reflected on the virus titer obtained from the culture supernatants of transfected cells (see FIG. 4B).

It is important that synthetic PRRSV RNA transcripts harboring an authentic 5′ and 3′ ends can be produced by in vitro run-off transcription reaction using a full-length infectious PRRSV cDNA as a template. A full-length PRRSV cDNA is essential for the production of infectious synthetic RNAs through in vitro transcription reaction (see FIG. 5). The present inventors synthesized RNA transcripts having an authentic 5′ ends by engineering the SP6 promoter transcription start in front of the virus genome. Previous studies on the construction of infectious PRRSV cDNAs used m⁷G(5′)ppp(5′)G cap structure analog for in vitro T7 polymerase transcription reaction (Nielsen et al., J. Virol. 77: 3702-3711, 2003; Meulenberg et al., J. Virol. 72: 380-387, 1998), and as a result, they produced synthetic PRRSV RNA transcripts containing one extra G nucleotide at their 5′ end (Contreras et al., Nucleic Acids Res. 10: 6353-6362, 1982). The present inventors confirmed that one extra G nucleotide incoporated during the transcription reaction using m⁷G(5′)ppp(5′)G cap structure analog did not affect the infectivity of the RNA transcripts (see FIG. 9A). Since the incoporation of m⁷G(5′)ppp(5′)A cap structure analog could produce capped synthetic RNA transcripts harboring the authentic 5′ end of the viral genome, the present inventors used the m⁷G(5′)ppp(5′)A cap structure analog, instead of the m⁷G(5′)ppp(5′)G. The importance of cap structure at the 5′ end of PRRSV becomes clear by the fact that no infectivity was detected in the cells transfected with full-length PRRSV RNA transcripts without cap structure (see FIG. 9A).

The 3′ end of PRRSV genomic RNA ends with poly (A) tail (Snijder and Meulenberg, Fields Virology, 4th ed. Lippincott Williams & Wilkins Publisher, Philadelphia, Pa., 2001; Cavanagh, Arch. Virol. 142: 629-633, 1997; Meulenberg et al., Virology 192: 62-72, 1993). The 3′ end of the genomic RNA of PRRSV PL97-1/LP1, the first Korean isolate, used in the present invention, has 54 nucleotides of the poly (A) tail. In order to investigate the role of the poly (A) tail in virus replication, Xba I restriction endonuclease recognition sequence was inserted in front of the poly (A) tail of infectious PRRSV cDNA pBAC/PRRSV/FL to be used as a run-off site, leading to the construction of pBAC/PRRSV/FLnop(A) (see FIG. 9B). The engineered pBAC/PRRSV/FLnop(A) cDNA was digested with AclI, then treated with mung bean nuclease, resulting in the linearized pBAC/PRRSV/FLnop(A)/AclI^{MBN}, which was used as a template for in vitro run-off transcription reaction to produce a full-length RNA transcript having the cap structure at its 5′ end but not having the poly (A) tail at its 3′ end. BHK-21 cells transfected with the RNA transcript not containing 54 poly (A) tail showed no infectivity at all, unlike those transfected with the polyadenylated RNA transcript synthesized from pBAC/PRRSV/FL/AclI^{MBN} (5.1×10⁵ PFU/μg) (see FIG. 9C).

The above results indicate that both cap structure at the 5′end of PRRSV viral genomic RNA and poly (A) tail at its 3′ end are essential for the generation of infectious PRRSV RNA transcripts.

V. The present invention provides a recombinant PRRSV obtained from the cells transfected with the RNA transcript synthesized from the PRRSV cDNA vector.

In the present invention, synthetic PRRSV was produced from the cells transfected with PRRSV RNA transcripts synthesized from the above PRRSV full-length infectious cDNA. Synthetic PRRSV produced by using four infectious cDNA templates (pBAC/PRRSV/FL/AclI^{MBN}, pBAC/PRRSV/FL/AclI, pBAC/PRRSV/FL/NotI and pBAC/PRRSV/FL/SdaI) was compared with parental virus PL97-1/LP1 used for the construction of the infectious PRRSV cDNA. As a result, a homogeneous population of large plaques was observed in the cells transfected with synthetic virus or with PL97-1/LP1 (see FIG. 6A). In addition, identical immunostaining pattern was also observed (see FIG. 6B). The growth properties showed that equivalent virus titers accumulated over time for all four synthetic viruses and the parental virus (see FIG. 6C). That is, the synthetic viruses recovered from the four infectious cDNA templates are phenotypically indistinguishable from the parental virus in terms of plaque morphology, cytopathogenicity, growth kinetics, and protein expression.

To confirm whether synthetic PRRSV was produced from a synthetic RNA transcript synthesized from infectious PRRSV cDNA through SP6 polymerase run-off transcription, a genetic marker was introduced into pBAC/PRRSV/FL by site-directed mutagenesis with PCR. Particularly, a silent point mutation, which does not alter any change in amino acids, was engineered in the ORF1a gene to create ClaI restriction endonuclease recognition site (pBAC/PRRSV/FLgm) (see FIG. 7A). In order to examine whether or not the novel ClaI genetic marker was recovered from the genomic RNA of synthetic PRRSV ‘PRRSV/FLgm/AclI^{MBN}’, the virus genomic RNA was extracted from PRRSV/FLgm/AclI^{MBN} virus and then amplified by RT-PCR. The amplified RT-PCR product was digested with ClaI, resulting in 902-bp and 1199-bp fragments. Thus, these results demonstrated that PRRSV/FLgm/AclI^{MBN} virus was originated from pBAC/PRRSV/FLgm/AclI^{MBN} cDNA. Therefore, this reverse genetics system using the infectious PRRSV cDNA of the present invention can be effectively used for direct molecular genetic studies on virus replication mechanisms of PRRSV and the development of a therapeutic agent and a genetic vaccine.

VI. The present invention provides a PRRSV expression vector containing the above PRRSV cDNA.

The present invention further provides a novel use of the infectious PRRSV cDNA and its derivative cDNAs as a novel heterologous gene expression vector applicable to a variety of eukaryotic cells.

Alphaviruses, which are also RNA viruses, can replicate in a variety of commonly used animal cells and thus have been successfully exploited as eukaryotic expression vectors in cell culture and in vivo (Agapov et al., Proc. Natl. Acad. Sci. 95: 12989-12994, 1998; Schlesinger, Mol. Biotechnol. 3: 155-165 1995). A full-length infectious PRRSV cDNA can be also used as an expression vector, that is, when a heterologous gene is inserted into the cDNA, recombinant PRRSV RNA transcript containing this foreign gene is synthesized through in vitro transcription reaction. In the cells transfected with the RNA transcript, the foreign gene of interest can be subsequently expressed (see FIGS. 10, 11, 12 and 13). Thus, the infectious PRRSV cDNA provided by the present invention can act as an expression vector for the rapid expression of a number of heterologous genes in a wide variety of eukaryotic cells.

VII. The present invention provides in variety of methods to express heterologous gene using the above PRRSV expression vector.

An expression vector delivers a heterologous target gene of interest to inside a cell for its expression. It was proved in the present invention that a full-length infectious PRRSV cDNA could be used as an expression vector for a heterologous gene of interest in various cells (see FIGS. 10, 11, 12 and 13). The present invention also explains a heterologous gene expression system based on a full-length infectious PRRSV cDNA as a bacterial artificial chromosome (BAC) (Yun et al., J. Virol. 77: 6450-6465, 2003). As a transient expression system, PRRSV provides several advantages such as (i) the recombinant virus is rapidly produced, (ii) it can replicate in a variety of eukaryotic cells upon transfection of synthetic RNAs, (iii) it is unable to infect humans, (iv) the genetically stable infectious cDNA is available and readily manipulated, and (v) the cytoplasmic replication of the RNA genome minimizes the possibility of integration and unwanted mutagenic consequences.

The present inventors also proved that the system using PRRSV to be used for the expression of a heterologous gene by two different ways. One way is related to a recombinant infectious PRRSV vector RNA and a recombinant PRRSV virus containing a heterologous gene and the other is related to a PRRSV viral replicon vector RNA, which is self-replicating and self-limited.

The method for the expression of a heterologous gene by using recombinant infectious PRRSV cDNA and viral replicon cDNA vectors of the present invention comprises the following steps:

1) Preparing a recombinant PRRSV cDNA expression vector by inserting a heterologous gene of interest into the infectious PRRSV cDNA or viral replicon cDNA vectors;

2) Preparing synthetic RNA transcripts from the above recombinant PRRSV cDNA expression vector;

3) Preparing transfectant cells by transfecting host cells with the above PRRSV RNA transcripts; and

4) Expressing the engineered heterologous gene of interest by culturing the transfectant cells.

The present inventors generated a recombinant full-length infectious PRRSV cDNA expressing EGFP (enhanced version of GFP) based on the above method (see FIG. 13A). BHK-21 cells were transfected with the synthetic RNA transcripts synthesized from the recombinant PRRSV cDNA, and then the expression of EGFP was confirmed therein (see FIG. 13B). The present inventors also produced recombinant infectious PRRSV viral particles containing the heterologous gene from the culture supernatant (see FIG. 13C).

The present inventors sought to construct a panel of self-replicating self-limited PRRSV viral replicons by using the infectious PRRSV cDNA pBAC/PRRSV/FL. The present inventors initially constructed a set of three viral replicons, designated as pBAC/PRRSV/RepLuc MB, pBAC/PRRSV/RepLuc ME, and pBAC/PRRSV/RepLuc DI, which have internal deletions of nt 12714-14194, nt 12163-14194, and nt 12163-15252, respectively (see FIG. 10). To facilitate the monitoring of viral replication, the present inventors also inserted at the site of each deletion the expression cassette containing the EMCV IRES-driven luciferase (LUC) gene. LUC was chosen as the reporter since its expression is easy to monitor in a highly quantitative and sensitive manner. The present inventors then examined whether the viral replicon RNAs derived from the three cDNA templates were replication-competent by monitoring the expression of the LUC gene after their transfection in BHK-21 cells. As a result, PRRSV/RepLuc MB and PRRSV/RepLuc ME viral replicon RNAs were competent in replication but PRRSV/RepLuc DI RNA was not. To determine the location of the minimal cis-acting element required for viral replication at the 3′ end of the viral genome, the present inventors constructed the eight viral replicons pBAC/PRRSV/RepLuc S1 to S8 by systematically deleting additional sequences towards the 3′ end of pBAC/PRRSV/RepLuc ME (see FIG. 10A). Of the eight viral replicons, only PRRSV/RepLuc S8 RNAs were replication-competent (see FIG. 10B). Thus, 8 viral replicons not containing 911 nucleotides from the 3′ end of the viral genome were all incapable of replication. On the other hand, virus replicons containing at least 911 nucleotides from the 3′ end were capable of replication (see FIG. 10). The above results indicate that a cis-acting element is located within the PRRSV ORF7 coding region (see FIGS. 10, 11, 12 and 13).

VIII. The present invention provides PRRSV 5′ mutant cDNAs, which are lacking 1-15 nucleotides at the 5′ end of the viral genome.

Eight constructs (pBAC/PRRSV/FL/Δnt1, pBAC/PRRSV/FL/Δnt3, pBAC/PRRSV/FL/Δnt5, pBAC/PRRSV/FL/Δnt7, pBAC/PRRSV/FL/Δnt9, pBAC/PRRSV/FL/Δnt11, pBAC/PRRSV/FL/Δnt13, and pBAC/PRRSV/FL/Δnt15) were designed to lack 1, 3, 5, 7, 9, 11, 13, and 15 nucleotides respectively, from the 5′ end of the genomic PRRSV RNA(see FIG. 14). After construction of the above 8 constructs, the present inventors generated synthetic RNA transcripts derived from each mutant cDNA construct to investigate their specific infectivity therein. As a result, when consecutive nucleotides were deleted at the 5′ end of PRRSV genomic RNA, specific infectivity was reduced or completely abolished (see FIG. 15).

The present inventors further examined the morphology of plaques and foci recognized by immunostaining with a mouse anti-ORF7 Mab. As a result, a relatively homogeneous population of large plaques and foci were observed in the cells transfected with pBAC/PRRSV/FL/←nt1- and pBAC/PRRSV/FL/Δnt3-drived synthetic RNAs, as seen in the cells transfected with the wild-type infectious RNA (see FIG. 15). On the contrary, a relative heterogeneous population of small plaques and foci were observed in the cells transfected with pBAC/PRRSV/FL/Δnt5- and pBAC/PRRSV/FL/Δnt7-drived synthetic RNAs (see FIG. 15). Neither infectivity nor plaque/focus was detected in the cells transfected with the synthetic RNAs having 9 or more nucleotide deletion at the 5′ end of the viral genomic RNA (see FIG. 15).

IX. The present invention provides PRRSV 5′ mutant cDNAs and their pseudorevertant viruses whose infectivity is recovered by adding various sizes of novel nucleotides to their 5′ ends. At this time, the novel nucleotides are preferred to be AT-rich and to be selected from a group consisting of TATG, AAG, ATTATA, TATTATA, ATTATAT, TATTATAT, TATCATAT, ATATATATAT, ATATATATATAT and ATTTATAT.

It should be noted that two mutants harboring the deletion of 5 and 7 nucleotides produced plaques of heterogeneous sizes, indicating some instability. This was more evident when supernatants harvested from the transfected cells were passaged once on naïve MARC-145 cells. These passaged pseudorevertants derived from the mutant cDNAs containing 1-, 3-, 5-, and 7-nucleotide deletions produced similar amounts of the PRRSV Nsp1a protein upon infection with the same amount of the viruses, as compared to the wild-type virus (see FIG. 16). Furthermore, their growth kinetics (see FIG. 17) and plaque morphology (see FIG. 18) were very similar, with a relatively homogeneous population of large plaques observed in the cells infected with each of these pseudorevertants, including the 5- and 7-nucleotide deletion mutants.

As shown in FIG. 19, 33 of 42 independent clones obtained from the pBAC/PRRSV/FL/Δnt1-derived pseudorevertants appeared to be converted to the wild-type virus by the acquisition of one A nucleotide at the site of the deletion. The remainder of the 9 clones had the same sequence as pBAC/PRRSV/FL/Δnt1 (see FIG. 19). In case of the pBAC/PRRSV/FL/Δnt3-derived pseudorevertants, 32 out of 57 independent clones had acquired three nucleotides (ATG) at the utmost 5′ end, which render the sequence identical to the wild-type virus (see FIG. 19). Twenty-one clones contained an insertion of 4 (TATG) while four clones appeared to contain an insertion of 3 (AAG) nucleotides at the deletion site (see FIG. 19). Interestingly, the pBAC/PRRSV/FL/Δnt5-derived pseudorevertants were found to have a deletion of the 5′-end single G nucleotide, the first nucleotide in this mutant construct. Moreover in 39 of 49 independent clones there was an insertion of 6 novel (ATTATA) nucleotides at the deletion site while 10 clones contained a 7-nucleotide insertion (TATTATA) (see FIG. 19). For the pBAC/PRRSV/FL/Δnt7-derived pseudorevertants, the acquisition of novel 5′ end sequences was found to be more heterogeneous than the mutants described above. Specifically, a majority of the sequenced clones (28/48 and 9/48 clones) had an insertion of 7 (ATTATAT) and 8 (TATTATAT) nucleotides at the site of the deletion, respectively, and appeared to be identical to two of the pBAC/PRRSV/FL/Δnt5-derived pseudorevertants (see FIG. 19). In addition, 4 independent clones had 8 novel nucleotides (TATCATAT) inserted at the deletion site while 2 further clones had the 8-nucleotide sequence ATTTATAT inserted at this site (see FIG. 19). In addition, 3 and 2 independent clones appeared to have 5 and 6 AT repeats at the site of the deletion, respectively (see FIG. 19). Overall, these pseudorevertants appeared to have acquired novel 5′ sequences of various sizes, composed mainly of A and T bases.

To address the importance of these novel 5′ sequences, the present inventors reconstructed 8 derivatives of the PRRSV 5′-end truncated mutants with all of these novel sequences and determined the specific infectivities of their RNA transcripts (see FIG. 20). In all reconstructed cases, their specific infectivities were increased to a level similar to that of the wild-type (see FIG. 20). Among all of the pseudorevertants, the present inventors found three cases that were not reconstructed, since the resulting mutations were identical to either the wild-type (PRRSV/FL/Δnt1/Rev1 and PRRSV/FL/Δnt3/Rev1) or the original truncated mutant (PRRSV/FL/Δnt1/Rev2) (see FIG. 20). According to plaque/focus morphology, the cells transfected with four synthetic RNAs (derived from pBAC/PRRSV/FL/Δnt3/Rev2, pBAC/PRRSV/FL/Δnt7/Rev1, pBAC/PRRSV/FL/Δnt7/Rev2, and pBAC/PRRSV/FL/Δnt7/Rev6) formed a homogeneous population of large plaques/foci, as seen with the wild-type infectious cDNA (see FIG. 20). In addition, the present inventors also observed a homogeneous population of medium (pBAC/PRRSV/FL/Δnt3/Rev3 and pBAC/PRRSV/FL/Δnt7/Rev3) and small (pBAC/PRRSV/FL/Δnt7/Rev4 and pBAC/PRRSV/FL/Δnt7/Rev5) sized plaques/foci (see FIG. 20). These results demonstrated that the addition of novel AT-rich sequences to the utmost 5′ end of the PRRSV 5′ deletion mutants, but not changes elsewhere in their genomes, allowed efficient PRRSV replication.

The above results plainly showes the importance of the PRRSV 5′ end nucleotide sequence ¹ATGACGT⁷ in RNA replication. Several novel AT-rich PRRSV 5′ sequences detected in the pseudorevertants were able to functionally replace the deleted ¹ATGACGT⁷. Although the functional role of these novel sequences is not completely understood, the complementary sequence of each of these novel 5′ sequences at the utmost 3′ end of negative-sense RNA is predicted to be involved in the initiation of positive-sense RNA synthesis.

In conclusion, the present invention contains a full-length PRRSV genomic RNA, a genetically stable full-length infectious PRRSV cDNA BAC clone, and its derivatives including recombinant infectious cDNAs and a number of viral replicons. Thus, the present invention not only offers a means of directly investigating the molecular mechanisms of PRRSV replication and pathogenesis, it can also be used to generate new heterologous gene expression vectors and genetically defined antiviral vaccines. Furthermore, the present invention can also be effectively used for the development of a therapeutic agent, a vaccine, a diagnostic agent, and a vector for the expression of a heterologous foreign gene in cells, in vivo and in vitro, for the DNA immunization and for the temporary gene therapy.

### PREFERRED EMBODIMENTS

Practical and presently preferred embodiments of this invention are illustrated in the following Examples.

However, it will be appreciated that those skilled in the art, on consideration of this disclosure, may make modifications and improvements within the spirit and scope of the present invention.

### Example 1

### Isolation and purification of PRRSV

### <1-1> Cells and Viruses

MARC-145 cells were maintained in minimum essential medium (MEM) containing 5% fetal bovine serum (FBS), nonessential amino acids, sodium pyruvate, and antibiotics in 5% CO₂ at 37° C. BHK-21 cells were grown in α-MEM supplemented with 10% FBS, 2 mM L-glutamine, vitamins, and antibiotics in 5% CO₂ at 37° C. All reagents used in cell culture were purchased from Life Technologies, Inc., Gaithersburg, Md. The parental PRRSV used in the present invention is the first Korean PRRSV strain, PL97-1, which was isolated in 1997 from the serum of an infected pig. High-titer virus stocks were obtained by cultivation in MARC-145 cells at a low multiplicity of infection (MOI) of 0.1 for 72 hr. The viruses were then clarified by centrifugation (2,000 rpm for 10 min), aliquotted, and stored at −80° C. until use.

Virus titers were determined by the plaque assay using MARC-145 cells. Particularly, the cells were pre-seeded in a six-well plate at a density of 3×10⁵ per well for 12-18 hr and then infected with serial 10-fold dilutions of virus for 1 hr at 37° C. with frequent agitation. The cell monolayers were then overlaid with MEM containing 0.5% SeaKem LE agarose (FMC BioProducts, Rockland, Me.) and 5% FBS and incubated for 4 days at 37° C. with 5% CO₂. The resulting plaques were visualized by fixation with 7% formaldehyde followed by staining with crystal violet (1% [w/v] in 5% ethanol).

### <1-2> Plaque Purification

PL97-1-infected MARC-145 cells were incubated with agarose as for the virus titration and virus clones were isolated by picking individual plaques with sterile Pasteur pipettes. The viruses were eluted from the agarose in 1 ml of medium by slow rocking at 4° C. for 1 hr, amplified once by cultivation in MARC-145 cells and stored at −80° C.

When a monolayer of susceptible MARC-145 cells was infected with the first Korean PRRSV strain PL97-1, while homogeneous large plaques predominated, a minority of relatively small plaque-forming variants was also observed (FIG. 1A). To obtain a genetically homogeneous population of large plaque-forming viruses, the present inventors cloned three large plaque-forming viruses by plaque-purification on MARC-145 cells. These were designated as PL97-1/LP1, 2, and 3. Of these, PL97-1/LP1 consistently maintained its large plaque phenotype during virus amplification and cultivation on MARC-145 cells (FIG. 1A) and produced a high virus titer of ≈10⁵ PFU/ml by 72 hr post-infection.

### <1-3> Investigation of PRRSV Protein Expression by Immunofluorescence Assays

MARC-145 cells (1×10⁵) were pre-seeded in a 4-well chamber slide for 12 hours, and then mock-infected or infected for 36 hours with a MOI of 1 of the original PRRSV PL97-1 strain or the PRRSV PL97-1/LP1 isolate. PRRSV ORF7 was immunostained by first fixing the cells in phosphate-buffered saline (PBS) containing 0.37% (v/v) formaldehyde for 30 min at 25° C., washing them three times with PBS and permeabilizing them in PBS containing 0.2% (v/v) Triton X-100 for 10 min at 37° C. The cells were then washed four times with PBS, rehydrated in PBS for 15 min, and blocked in PBS containing 5% (w/v) BSA for 1 hr at 37° C. Thereafter, the cells were incubated for 2 hr at 25° C. with mouse anti-ORF7 Mab (6D7/D2) at a 1:1000 dilution, washed with PBS three times, and incubated for 2 hr at 25° C. with FITC-conjugated goat anti-mouse IgG (Jackson ImmunoResearch Labs Inc., West Grove, Pa.) at a 1:1000 dilution. After washing the cells with PBS three times again, the cells were incubated in PBS containing 5 μg/ml propidium iodide and 5 μg/ml RNase A for 30 minutes at 37° C. to localize the nuclei. They were then mounted with 0.2 ml of 80% glycerol. Images were acquired with a Zeiss Axioskop confocal microscope equipped with a 63× objective using a Bio-Rad MRC 1024 and LaserSharp software.

As a result, immunostaining of the PRRSV ORF7 protein in MARC-145 cells infected with PL97-1/LP1 or the parental PL97-1 revealed bright green fluorescence that predominated around the perinucleous membrane (FIG. 1B). In both cases, speckle fluorescence staining within the nucleolus that indicates the nucleolar localization of the PRRSV ORF7 protein was also observed, as previously described (Rowland et al., Virology 316: 135-145, 2003; Rowland and Yoo, Virus Res. 95: 23-33, 2003; Yoo et al., J. Virol. 77: 12173-12183, 2003). As expected, no green fluorescence was observed in the mock-infected cells (FIG. 1B). Based on the above results, the present inventors chose PL97-1/LP1 as the PRRSV parental virus in the construction of an infectious cDNA molecular clone.

### Example 2

### Determination of the Complete Nucleotide Sequence of PRRSV PL97-1/LP1 Genomic RNA

Viral RNA was extracted from 100 μl of the virus stock with 300 μl of TRIzol LS reagent according to the manufacturer's instructions (GIBCO/BRL, Gaithersburg, Md.). To ensure consistent recovery of the extracted viral RNA, 5 μg glycogen (Boehringer Mannheim, Indianapolis, Ind.) was added to the extracted samples as a carrier prior to precipitation with isopropanol. According to the previously established method, the sequence of the entire PRRSV genomic RNA was identified (FIG. 2).

The extracted viral RNA served as a template for four cDNA synthesis reactions that generated four long overlapping cDNAs (Fr1-4) that spanned the entire viral RNA genome apart from the 5′ and 3′ termini. These reverse transcription reactions employed the Superscript II RNaseH(−) RT system (GIBCO/BRL): thus, 10 μl extracted viral RNA was incubated at 37° C. for 1 hr in a 20 μl reaction mixture containing the RT buffer supplied by the manufacturer, 5 pmol of the PR1RT, PR2RT, PR3RT, or PR4RT primer represented by SEQ. ID. No 1, No 2, No 3 or No 4, 100 U Superscript II RT, 40 U RNaseOUT, 0.1 mM DTT, and 10 mM dNTP mix. PCR was then used to amplify 5 μl of the four cDNA products using the low-error-rate Pyrobest Pfu DNA polymerase (Takara Bio Inc., Shiga, Japan) and the PR1Fz (SEQ. ID. No 5)+PR1Rz (SEQ. ID. No 6), PR2Fz (SEQ. ID. No 7)+PR2Rz (SEQ. ID. No 8), PR3Fz (SEQ. ID. No 9)+PR3Rz (SEQ. ID. No 10) and PR4Fz (SEQ. ID. No 11)+PR4Rz (SEQ. ID. No 12) primer pairs, respectively. The PCR reaction consisted of 35 cycles of denaturation (94° C. for 30 sec), annealing (60° C. for 30 sec), and extension (72° C. for 6 min), with a final extension step (72° C. for 10 min). These reactions resulted in the Fr1 (nt 180-5297), Fr2 (nt 3708-9108), Fr3 (nt 7570-13051), and Fr4 (nt 9610-15238) amplicons, respectively.

To sequence the 5′-terminus of the RNA genome, the present inventors adopted a 5′RACE protocol with a minor modification. First-strand cDNA was first synthesized by Superscript II RT from the viral RNA using the 5′-end-unphosphorylated primer PR50 (SEQ. ID. No 13). The RNA in the first-strand cDNA-RNA hybrid was then degraded in a 75 μl reaction mixture containing 60 U RNase H, 20 μl first-strand cDNA reaction mixture, and the buffer supplied by the manufacturer (Takara) at 30° C. for 1 hr. The resulting first-strand cDNA was phenol-extracted, precipitated with 100% ethanol, and resuspended in 14 μl RNase-free water. To introduce a primer-binding site, the 3′ end of the first-strand cDNA was ligated at 15° C. for 12 hr to the synthetic oligonucleotide PRX (SEQ. ID. No 14), which had been phosphorylated on its 5′-end and modified on its 3′ end by the incorporation of ddATP to prevent intra- and inter-molecular ligation, as described previously (Yun et al., J. Virol. 77: 6450-6465, 2003). The 40 μl ligation reaction mixture contained 40 U T4 RNA ligase, 7 μl single-stranded cDNA, 10 pmol PRX, 20% PEG #6000, and buffer (Takara). The PRX-ligated first-strand cDNA was then phenol-extracted, ethanol-precipitated, and resuspended in 20 μl RNase-free water. One-twentieth of this cDNA was PCR-amplified by using the PR49 (SEQ. ID. No 15)+PRXR (SEQ. ID. No 16) forward and reverse primers. The PCR reaction consisted of 30 cycles of denaturation (94° C. for 30 sec), annealing (60° C. for 30 sec), and extension (72° C. for 1 min), with a final extension step (72° C. for 10 min). The 334-bp PstI-SacI fragment of the resulting cDNA amplicons (FrF) was then inserted into the pRS2 vector that had been digested with the same enzymes. This generated pRS/PRRSV/FrF.

To sequence the 3′-terminus of the RNA genome, the present inventors adopted a 3′RACE protocol (Yun et al., J. Virol. 77: 6450-6465, 2003). Here, the 5′-phosphorylated and 3′-blocked PRX oligonucleotide was ligated at 15° C. for 12 hr to the 3′ end of the viral RNA to provide a specific primer-binding site for RT-PCR. The 20 μl ligation reaction contained 10 U T4 RNA ligase (New England Biolabs, Inc., Beverly, Mass.), 40 U RNaseOUT, 10 pmol PRX, extracted viral RNA, and the buffer supplied by the manufacturer. After the incubation, the PRX-ligated viral RNA was phenol-extracted, precipitated with 100% ethanol, and resuspended in 20 μl RNase-free water. Half portion of this was subsequently used for cDNA synthesis by using Superscript II RT and the PRXR primer, as described above. A quarter of the first-strand cDNA product was amplified by using the PR41 (SEQ. ID. No 17)+PRXR forward and reverse primers with 30 cycles of denaturation (94° C. for 30 sec), annealing (60° C. for 30 sec), and extension (72° C. for 2 min), with a final extension step (72° C. for 10 min). The 1537-bp NheI-PstI fragment of the cDNA amplicons (FrR) was then cloned into the XbaI- and PstI-digested pGEM3Z vector, thus generating pGEM/PRRSV/FrR.

As a result, the entire nucleotide sequence of PL97-1/LP1, represented by SEQ. ID. No 18, was identical to that of the parental virus PL97-1 except for three silent nucleotide substitutions, one in ORF1a (T⁴²³⁰→C), one in ORF1b (C¹⁰⁹⁷⁷→T), and one in ORF5 (T¹³⁹⁷⁶→A).

### Example 3

### Construction of the Full-Length Infectious PRRSV cDNA Using BAC

All plasmids were constructed by standard molecular biology procedures (Sambrook et al., a laboratory manual, 2nd ed. Cold Spring Harbor Laboratory, Cold Spring Harbor, N.Y., 1989). The four long overlapping cDNA amplicons Fr1-4 that were originally used to sequence the complete genome of PL97-1/LP1 were first subcloned into pBAC^{SP6}/JVFLx/XbaI (Yun et al., J. Virol. 77: 6450-6465, 2003). Thus, the 7734-bp SfiI-PacI fragment of PBAC^{SP6}/JVFLx/XbaI was ligated with the 5131, 5414, 5495, and 5642-bp SfiI-PacI fragments of the Fr1-4 amplicons to construct pBAC/PRRSV/Fr1, pBAC/PRRSV/Fr2, pBAC/PRRSV/Fr3 and pBAC/PRRSV/Fr4, respectively. Careful sequencing of these subclones, along with pRS/PRRSV/FrF (which was originally used to sequence the 5′ region of the PRRSV genome) and pGEM/PRRSV/FrR (which was originally used to sequence the 3′ region of the PRRSV genome) showed their nucleotide sequences were identical to that of the parental PL97-1/LP1 virus apart from two point mutations.

One was the C⁴⁵⁸¹→T (silent) substitution within the ORF1a gene in pBAC/PRRSV/Fr1. However, this mutation was not present in the overlapping region of pBAC/PRRSV/Fr2. By taking the cDNA fragment containing the correct C⁴⁵⁸¹ base from pBAC/PRRSV/Fr2, as described below, this mutation was not incorporated into the full-length cDNA. The other mutation was the G¹³³⁰⁴→A substitution in pBAC/PRRSV/Fr4 that would result in Thr substituting Ala at position 22 of ORF4. This mutation was corrected by PCR-based site-directed mutagenesis, wherein two fragments of pBAC/PRRSV/Fr4 that had been PCR-amplified with the PR40 (SEQ. ID. No 19)+PRcR (SEQ. ID. No 20) and PRcF (SEQ. ID. No 21)+PR4Rz primer pairs were fused by a second round of PCR with the PR40+PR4Rz primers. The 1099-bp BsrGI-SacII fragment of the resulting amplicons was ligated with the 7002-bp SacII-XhoI and 5275-bp XhoI-BsrGI fragments of pBAC/PRRSV/Fr4, resulting in pBAC/PRRSV/Fr4c.

To engineer the SP6 promoter immediately upstream of the PRRSV viral genome, the pRS/PRRSV/FrF subclone was modified. One fragment each of pBAC^{SP6}/JVFLx/XbaI and pRS/PRRSV/FrF was amplified by PCR with the JP41 (SEQ. ID. No 22)+PRI5Rsp6 (PRI5Rsp6 incorporates the antisense sequence of the SP6 promoter) (SEQ. ID. No 23) and PRI1F (SEQ. ID. No 24)+PR49 primer pairs, respectively. These two fragments were then fused by a second round of PCR with the JP41+PR49 primers. The 456-bp SpeI (T4 DNA polymerase-treated)-SacI fragments of these fused PCR amplicons were then ligated with the 2746-bp PstI (T4 DNA polymerase-treated)-SacI fragments of pRS/PRRSV/FrF to produce the pRS^{SP6}/PRRSV/FrF construct.

To introduce an artificial run-off site that would generate an authentic or close to authentic 3′ terminus on the viral genome that is formed during run-off transcription of the plasmid linearized at the 3, end of the viral genome, the present inventors modified pGEM/PRRSV/FrR so that the nucleotide sequence of the authentic 3′-terminus is followed by a row of unique restriction endonuclease recognition sites, namely, AclI, NotI, and SdaI. To do this, the viral RNA was first ligated with the 5′-phosphorylated oligonucleotide PR3endX (SEQ. ID. No 25), which bears the AclI, NotI, and SdaI sites in a row, by using the T4 RNA ligase as described above. The oligo-ligated viral RNA was subsequently used for cDNA synthesis using Superscript II RT and the PR3endY (SEQ. ID. No 26) primer, which is complementary to PR3endX. The first-strand cDNA was then PCR-amplified by using the PR41+PR3endY primers. The 1295-bp MluI-PstI fragment of these cDNA amplicons was then ligated to the 3429-bp Mlu I-Pst I fragment of pGEM/PRRSV/FrR to produce the pGEM^{ROS}/PRRSV/FrR subclone construct.

After constructing the six subclone plasmids pRS^{SP6}/PRRSV/FrF, pBAC/PRRSV/Fr1, pBAC/PRRSV/Fr2, pBAC/PRRSV/Fr3, pBAC/PRRSV/Fr4c, and pGEM^{ROS}/PRRSV/FrR, the present inventors assembled them into a full-length PRRSV cDNA in a stepwise manner, as illustrated in FIG. 2. First, the pBAC^{SP6}/PRRSV/FrF1 subclone was constructed by ligating together the 451-bp PacI-SacI fragment of pRS^{SP6}/PRRSV/FrF, the 4226-bp SacI-EagI fragment of pBAC/PRRSV/Fr1, and the 7455-bp PacI-EagI fragment of pBAC^{SP6}/JVFLx/XbaI. The pBAC/PRRSV/Fr23 subclone was then produced by ligating together the 3048-bp EagI-AvrII fragment of pBAC/PRRSV/Fr2, the 2256-bp AvrII-RsrII fragment of pBAC/PRRSV/Fr3, and the 14050-bp EagI-RsrII fragment of pBAC^{SP6}/JVFLx/XbaI. Next, the pBAC^{SP6}/PRRSV/FrF123 subclone was constructed by ligating together the 4677-bp PacI-EagI fragment of pBAC^{SP6}/PRRSV/FrF1, the 5304-bp EagI-RsrII fragment of pBAC/PRRSV/Fr23, and the 7449-bp PacI-RsrII fragment of PBAC^{SP6}/JVFLx/XbaI. The pBAC/PRRSV/Fr4cR subclone was then produced by ligating together the 4348-bp RsrII-MluI fragment of pBAC/PRRSV/Fr4c, the 1301-bp MluI-SphI fragment of pGEM^{ROS}/PRRSV/FrR, and the 10055-bp RsrII-SphI fragment of pBAC^{SP6}/JVFLx/XbaI. Finally, the full-length PRRSV cDNA pBAC/PRRSV/FL was assembled by ligating the 9981-bp PacI-RsrII fragment of PBAC^{SP6}/PRRSV/FrF123 and the 5649-bp RsrII-SphI fragment of pBAC/PRRSV/Fr4cR with the 7592-bp PacI-SphI fragment of pBAC/NADLJiv90-, which is an infectious cDNA of the bovine viral diarrhea virus strain NADLJiv90-.

In conclusion, pBAC/PRRSV/FL (SEQ. ID. No 27) was constructed in which the full-length PRRSV cDNA of an authentic PRRSV PL97-1/LP1 virus genome was cloned downstream of SP6 promoter (FIG. 2).

### Example 4

### Estimation of Specific Infectivities of the RNA Transcripts from an In Vitro Run-Off Transcription with the Full-Length PPRSV cDNA pBAC/PRRSV/FL <4-1>

### <4-1> Synthesis of Full-Length PRRSV RNA Transcripts Via In Vitro Run-Off Transcription

The present inventors estimated the specific infectivity of the synthetic RNAs that were transcribed in vitro from pBAC/PRRSV/FL. First, pBAC/PRRSV/FL was linearized by digestion with AclI, NotI, or SdaI to prepare three different cDNA templates. These were designated as pBAC/PRRSV/FL/AclI, pBAC/PRRSV/FL/NotI, and pBAC/PRRSV/FL/SdaI, respectively, and served as templates for SP6 polymerase run-off transcription in the presence of the m⁷G(5′)ppp(5′)A cap structure analog. As summarized in FIG. 3, all transcription reactions produced capped synthetic RNA transcripts with authentic 5′ ends (Contreras et al., Nucleic Acids Res. 10: 6353-6362, 1982). However, the 3′ ends varied depending on the template used in the transcription reaction. Transcription of the pBAC/PRRSV/FL/AclI template yielded synthetic RNAs containing two extra nucleotides (CG) of virus-unrelated sequence at its 3′ end as a result of copying the 5′ overhang left by the Acl I digestion (FIG. 3B). Transcription of the pBAC/PRRSV/FL/NotI and pBAC/PRRSV/FL/SdaI templates also produced synthetic RNAs containing 10 (CGTTGCGGCC) or 14 (CGTTGCGGCCGCCC) extra nucleotides of virus-unrelated sequence at their 3′ ends, respectively (FIG. 3B). In some flaviviruses, unrelated sequences at the 3′-end of synthetic RNAs transcribed from an infectious cDNA have been reported to diminish or abrogate their specific infectivity (Yun et al., J. Virol. 77: 6450-6465, 2003; Yamshchikov et al., Virology 281: 272-280, 2001). Thus, the present inventors sought to generate a synthetic RNA bearing the authentic 3′-end of the PRRSV genome. This was achieved by linearizing pBAC/PRRSV/FL with AclI followed by mung bean nuclease (MBN) treatment to remove the 5′ overhang left by the AclI digestion. The resulting construct was designated as PBAC/PRRSV/FL/AclI^{MBN} (FIG. 3B).

In vitro transcription from these template cDNAs was carried out at 37° C. for 1 hr with 100-200 ng of the template cDNA in a 25-μl reaction mixture containing 0.6 mM cap structure analog [m⁷G(5′)ppp(5′)A or m⁷G(5′)ppp(5′)G, NEB Inc.], 0.5 μM [3H]UTP (1.0 mCi/ml, 50 Ci/mmol, New England Nuclear Corp., Boston, Mass.), 10 mM DTT, 1 mM each UTP, GTP, CTP, and ATP, 40 U of RNaseOUT, 15 U of SP6 RNA polymerase (GIBCO/BRL), and the buffer supplied by the manufacturer. The template cDNAs were then digested with 4 U of RNase-free DNase I (Ambion, Inc., Austin, Tex.) and the RNA transcripts were purified by phenol-chloroform extraction and ethanol precipitation and quantified on the basis of [³H]-UTP incorporation, as measured by RNA adsorption to DE-81 (Whatman, Maidstone, UK) filter paper (Sambrook et al., a laboratory manual, 2nd ed. Cold Spring Harbor Laboratory, Cold Spring Harbor, N.Y., 1989). One twentieth of the reaction mixtures were examined by agarose gel electrophoresis to ensure the integrity of the RNA transcripts.

<4-2> Transfection with the Full-Length PRRSV RNA Transcripts

To transfect cells with the synthetic RNA transcripts prepared above, electroporation was performed using ECM 830 electroporator (BTX Inc., San Diego, Calif.) according to the manufacturer's instruction. MARC-145 or BHK-21 cells were pre-seeded at a density of 2 or 3×10⁶ cells per p150 culture dish for 24 hr at 37° C. with 5% CO₂. The subconfluent cells were then trypsinized and washed three times with ice-cold RNase-free PBS. After, resuspension at a density of 2×10⁷ cells/ml in PBS, 400 μl of the cells were mixed with 2 μg of synthetic RNA in a cuvette with a gap width of 0.2 cm. The cells were electroporated with 10 (MARC-145 cells) or 5 (BHK-21 cells) pulses of current, respectively, by using an ECM 830 electroporator (BTX Inc., San Diego, Calif.) set at 900 V and 99-μs pulse length. The cells were then transferred to 10 ml of fresh media.

### <4-3> Specific Infectivity of the Full-Length PRRSV RNA Transcripts

Infectious center assays were used to quantitate the specific infectivity of synthetic RNA transcripts. Thus, the electroporated cells were serially diluted 10-fold and plated onto monolayers of untransfected MARC-145 cells (3×10⁵) in a 6-well plate. After 6 hr cultivation, the cell monolayers were then overlaid with MEM containing 0.5% SeaKem LE agarose incubated for 4-5 days at 37° C. with 5% CO₂. The resulting plaques were visualized by staining with crystal violet.

Transfection of the synthetic RNA transcripts into susceptible MARC-145 cells by using our optimized electroporation conditions indicated that the specific infectivities of the transcripts produced from pBAC/PRRSV/FL/AclI^{MBN}, pBAC/PRRSV/FL/AclI and pBAC/PRRSV/FL/NotI were similar at 3.3, 2.3 and 2.2×10⁴ PFU/μg, respectively (FIG. 4A). Thus, these synthetic RNAs are highly infectious. However, the specific infectivity of the transcripts from pBAC/PRRSV/FL/SdaI was ≈20-fold lower at 1.3×10³ PFU/g (FIG. 4A). Thus, reconstitution of the authentic 3′ end of the PRRSV genome is important for the generation of highly infectious synthetic PRRSV RNA transcripts.

While the present inventors were optimizing our electroporation conditions using MARC-145 cells, the inventors discovered that only #5% of these cells were competent in RNA uptake. This led the present inventors to examine the specific infectivity of the four synthetic RNAs by using BHK-21 cells, under the condition that >90% of the transfected cells were consistently competent for RNA uptake (Yun et al., J. Virol. 77: 6450-6465, 2003). BHK-21 cells are not susceptible to PRRSV infection. Thus, for the infectious center assays, the BHK-21 cells were transfected with the PRRSV RNA transcripts and placed onto a monolayer of MARC-145 cells. The infectivities of the PRRSV/FL/AclI^{MBN}, PRRSV/FL/AclI, PRRSV/FL/NotI, and PRRSV/FL/SdaI RNAs produced by the BHK-21 cells were significantly higher at 7.5×10⁵, 7.3×10⁵, 5.6×10⁵, and 8.6×10⁴ PFU/μg, respectively (FIG. 4A). Thus, the infectivities of the synthetic PRRSV RNA transcripts produced by BHK-21 cells were #20-fold higher than those produced by MARC-145 cells, which indicates that the system employing BHK-21 cells may be superior to MARC-145 cells in the characterization of viral mutants whose infectivities have been lost or reduced.

The specific infectivities of all four RNA transcripts reflected the virus titers in the culture supernatants of the transfected MARC-145 cells. At 24 hr post-transfection, when none of the transfected cells showed signs of a cytopathic effect (CPE), the titers from cells transfected with PRRSV/FL/AclI^{MBN}, PRRSV/FL/AclI, and PRRSV/FL/NotI ranged from 1.0-2.0×10³ PFU/ml (FIG. 4B). At 48 hr post-transfection, at which point the transfected cells clearly displayed CPE, these titers had increased ≈5-10-fold to 5.0-9.0×10³ PFU/ml (FIG. 4B). At 72 hr post-transfection, when most cells showed strong CPE and detached from the culture dishes, these titers had increased further to 5.0-8.0×10⁴ PFU/ml (FIG. 4B). However, with regard to the titers produced by the PRRSV/FL/SdaI RNA-transfected cells, at 24, 48 and 72 hrs post-transfection, only 40, 1.6×10² and 3.0×10⁴ PFU/ml were generated, respectively (FIG. 4B). Thus, cells transfected with synthetic RNA transcripts containing the authentic 3′ end rapidly produce high titers of PRRSV virus, although 2-10 extra nucleotides, but not 14, of virus-unrelated sequence at the 3′ end are also tolerated. Thus, PRRSV reverse genetics system of the present invention is highly efficient.

To confirm that the specific infectivity observed is due to infectious RNAs produced by the full-length PRRSV cDNA template, RNAs produced by SP6 polymerase run-off transcription from pBAC/PRRSV/FL/AclI^{MBN} cDNA in the presence or absence of DNase I were subsequently treated with DNase I or RNase A. The present inventors then analyzed the specific infectivities of these RNAs (FIG. 5). This showed that the cDNA template alone is not infectious (FIGS. 5A and 5B, Without SP6 Pol) but that the intact cDNA template is needed during the transcription reaction since DNase I treatment during the reaction abolished the infectivity (FIGS. 5A and 5B, DNase I During). DNase I introduced after the transcription reaction had no effect (FIGS. 5A and 5B, DNase I After) relative to the intact reaction mixture (FIGS. 5A and 5B, Without Treatment); however, RNase A treatment abolished the infectivity of the RNAs (FIGS. 5A and 5B, RNase A After). Thus, the PRRSV cDNA template per se is clearly not infectious but is needed to generate highly infectious synthetic RNA transcripts.

### Example 5

### Identification of the Synthetic PRRSVs Derived from the Infectious cDNAs Against the Parental Virus

The present inventors compared the synthetic PRRSVs recovered from the four infectious cDNA templates with the parental virus PL97-1/LP1. Cells were infected with synthetic PRRSVs recovered from the infectious cDNA templates and the parental virus PL97-1/LP1, and then the cell monolayers were overlaid with MEM containing 0.5% SeaKem LE agarose (FMC BioProducts, Rockland, Me.) and 10% FBS and incubated for 3-4 days at 37° C. with 5% CO₂. The resulting plaques were visualized by fixation with 3.7% formaldehyde for 4 hr at room temperature, followed by staining with crystal violet. Infection of MARC-145 cells with the synthetic viruses or with PL97-1/LP1 both resulted in homogeneous large plaques (FIG. 6A). The immunostaining patterns of the cells infected with either of the four synthetic viruses or PL97-1/LP1 were also identical (FIG. 6B). No PRRSV-specific fluorescence signal was observed in mock-infected cells (FIG. 6B).

The growth properties of the synthetic viruses were also investigated by infecting MARC-145 cells with the synthetic or parental viruses at an MOI of 1 and analyzing the virus titers in the media periodically (FIG. 6C). Equivalent virus titers accumulated over time for all four synthetic viruses and the parental virus. Similar growth kinetics were also obtained with low (0.1) and high (10) MOIs. Thus, the synthetic viruses recovered from the four infectious cDNA templates are phenotypically indistinguishable from the parental virus in plaque morphology, cytopathogenicity, growth kinetics, and protein expression.

The present inventors sequenced the 5′ and 3′ ends of the four synthetic PRRSV viral genomes and found that, as expected, their 5′ ends is identical to the 5′ end of the parental virus. With regard to the 3′ end, the present inventors found that the genomic RNAs of all four synthetic viruses were terminated with a poly (A) tail and did not retain the 2, 10, or 14 extra nucleotides of virus-unrelated sequence at their 3′ ends. Thus, the genomes of the viruses recovered from the four infectious cDNA templates bear authentic 5′ and 3′ ends. These observations validate the use of the infectious PRRSV cDNA of the present invention for direct molecular genetic analyses.

### Example 6

### Confirming the Assembly of Synthetic PRRSVs through the RNA Transcripts Derived from an SP6 Polymerase Transcription with an Infectious Full-Length PRRSV cDNA as the Template

<6-1> Inducing Silent Point Mutations into the Full-Length PRRSV cDNA

To confirm that synthetic PRRSV viruses were recovered from RNA transcripts synthesized from the cDNA by SP6 polymerase run-off transcription and rule out the possibility of contamination of the transfected cell medium from the parental virus, the present inventors introduced by PCR-based site-directed mutagenesis a genetic marker (gm) into pBAC/PRRSV/FL construct: namely, a silent point mutation (C²¹⁸⁷→T) in the ORF1a gene that generates a new ClaI restriction endonuclease recognition site. More specifically, a fragment of pBAC/PRRSV/FL was amplified by PCR with the PR3364 (SEQ. ID. No 28)+PR2172 (SEQ. ID. No 29) primers. The PR2172 primer contains the C²¹⁸⁷→T substitution that generates the ClaI site. The 1138-bp MluI-SphI fragment of the resulting amplicons was then ligated with the 12167-bp SphI-NotI and 9913-bp NotI-MluI fragments of pBAC/PRRSV/FL to produce the pBAC/PRRSV/FLgm construct (SEQ. ID. No 30) (FIG. 7A). Infectious PRRSV/FLgm/AclI^{MBN} viruses generated by the RNAs transcribed from the AclI-linearized MBN-treated pBAC/PRRSV/FLgm/AclI^{MBN} template did not differ phenotypically from the PRRSV/FL/AclI^{MBN} viruses. Thus, the C²¹⁸⁷→T substitution did not have a deleterious effect on viral growth.

The recovered PRRSV/FLgm/AclI^{MBN} viruses were then serially passaged in MARC-145 cells at an MOI of 0.1, and at each passage, the harvested viruses were treated with RNase A and DNase I to eliminate the possibility of carryover of input RNA transcripts and cDNA template (Yun et al., J. Virol. 77: 6450-6465, 2003; Mendez et al., J. Virol. 72: 4737-4745, 1998). To determine whether the new ClaI genetic marker was presented in the genome of PRRSV/FLgm/AclI^{MBN} viruses, viral RNAs from the PRRSV/FLgm/AclI^{MBN} viruses collected at passages 1 and 3 were subjected to RT-PCR with the PR1282 (SEQ. ID. No 31) and PR3364 primers that amplified a 2101-bp product encompassing the C²¹⁸⁷→T point mutation. The product was then digested with ClaI. The product from PRRSV/FLgm/AclI^{MBN} virus was divided into two fragments of 902-bp and 1199-bp but that from PRRSV/FL/AclI^{MBN} virus was not digested (FIG. 7B). Thus, the recovered PRRSV/FLgm/AclI^{MBN} virus originates from the pBAC/PRRSV/FLgm/AclI^{MBN} cDNA template.

### Example 7

### Confirming the Genetic Stability of the Infectious PRRSV cDNA After Multiple Passages

Since future molecular genetic studies of PRRSV using the infectious PRRSV cDNA rely on its genetic stability during manipulation (Yun et al., J. Virol. 77: 6450-6465, 2003), the present inventors extensively investigated the genetic structure and functional integrity of pBAC/PRRSV/FL. Briefly, two independent pBAC/PRRSV/FL-containing E. coli DH10B clones were grown in 10 ml of 2xYT containing 12.5 μg/ml chloramphenicol at 37° C. overnight. These primary cultures were maintained for 12 days by 10⁶-fold serial dilution every day. After every three passages, a large-scale sample of infectious cDNA plasmid was prepared by the SDS-alkaline method and purified by cesium chloride density gradient centrifugation (Sambrook et al., a laboratory manual, 2nd ed. Cold Spring Harbor Laboratory, Cold Spring Harbor, N.Y., 1989). The genetic stability of the plasmid DNA was monitored by assessing its restriction endonuclease pattern (which determined its genetic structure) and by measuring the specific infectivity of the synthetic RNA transcribed from the cDNA template (which determined its functional integrity).

E. coli DH10B was transformed with pBAC/PRRSV/FL and cultured on semisolid media at 37° C. This resulted in the appearance of homogeneous populations of small bacterial colonies harboring the infectious cDNA after 15-20 hr. Extensive restriction analysis of eight randomly picked clones showed no evidence of deletions or rearrangements and SP6 polymerase run-off transcription from all eight clones and transfection of BHK-21 cells consistently yielded synthetic RNAs with high specific infectivities ranging from 5.8-7.1×10⁵ PFU/μg.

Two independent pBAC/PRRSV/FL-containing E. coli DH10B clones were also grown in liquid media overnight and propagated for 12 days by serial daily 10⁶-fold dilutions (Yun et al., J. Virol. 77: 6450-6465, 2003; Almazan et al., Proc. Natl. Acad. Sci. 97: 5516-5521, 2000). In the present invention, each passage represented approximately 20 generations, which is consistent with previous reports (Yun et al., J. Virol. 77: 6450-6465, 2003; Almazan et al., Proc. Natl. Acad. Sci. 97: 5516-5521, 2000). Restriction analysis of the two independent cDNA plasmids at passages 0, 3, 6, 9, and 12 revealed no visible differences, even between passages 0 and 12. Thus, the two PRRSV cDNA clones were structurally highly stable, at least within the resolution of agarose gel electrophoresis analysis. Furthermore, the infectivity of the RNA transcripts from the two independent cDNA clones was unchanged at passages 0 to 12 (>5.0×10⁵ PFU/μg) (FIG. 8). Thus, the functional integrity of the infectious cDNA remained stable during serial growth in E. coli.

### Example 8

### Confirming the Requirements for the 5′ Cap Structure and 3′ poly (A) Tail of the PRRSV Genome in Viral Replication

To examine the role of the cap structure at the 5′ end of the viral genome in viral replication, run-off transcription from pBAC/PRRSV/FL/AclI^{MBN} cDNA template was performed in the presence or absence of the m⁷G(5′)ppp(5′)A or m⁷G(5′)ppp(5′)G cap structure analog. BHK-21 cells were electroporated with the synthetic RNAs and infectious center assay was carried out. SP6 polymerase run-off transcription in the absence of a cap structure analog produced full-length uncapped RNA transcripts but infectious center assays on BHK-21 cells showed that they were not infectious at all (FIG. 9A). In contrast, the RNA transcripts capped with m⁷G(5′)ppp(5′)A had a specific infectivity of 5.3×10⁵ PFU/μg (FIG. 9A). Thus, the cap structure is essential for PRRSV replication. In addition, the present inventors found that the capping of RNA transcripts with the m⁷G(5′)ppp(5′)G cap structure analog, which adds an extra G at the 5′ end of the viral genome, resulted in transcripts that were as infectious as those capped with m⁷G(5′)ppp(5′)A (FIG. 9A).

To examine the role of the poly (A) tail in viral replication, the pBAC/PRRSV/FL cDNA was modified by introducing a unique Xba I site immediately upstream of the poly (A) tail. This served as the run-off site and generated pBAC/PRRSV/FLnop(A) (SEQ. ID. No 32) (FIG. 9B) To construct the pBAC/PRRSV/FLnop(A) construct, a fragment of pBAC/PRRSV/FL was PCR-amplified with the PR37 (SEQ. ID. No 33)+PRnop(A) (SEQ. ID. No 34) primers. PRnop(A) is complementary to a sequence immediately upstream of the poly (A) stretch. The 1223-bp MluI-NotI fragment of the resulting amplicons was then ligated with the 17584-bp NotI-RsrII and 4348-bp RsrII-MluI fragments of pBAC/PRRSV/FL. SP6 polymerase run-off transcription of the Xba I-linearized MBN-treated cDNA template pBAC/PRRSV/FLnop(A)/XbaI^{MBN} produced capped RNA transcripts terminated with -GCC GAA ATT¹⁵⁴¹¹ that lacked the 54 poly (A) nucleotides. These unpolyadenylated RNAs were not infectious at all upon transfection into BHK-21 cells (FIG. 9C), unlike the polyadenylated RNAs transcribed from pBAC/PRRSV/FL/AclI^{MBN} (5.1×10⁵ PFU/μg). Thus, both the cap structure and poly (A) tail are essential for PRRSV replication.

### Example 9

### Confirmation of a Cis-Acting Element Required for PRRSV Self-Replication

### <9-1> Construction of PRRSV Viral Replicons

The present inventors constructed a panel of 11 PRRSV viral replicon vectors that express the luciferase (LUC) gene as a reporter (FIG. 10). To facilitate this construction, the 5649-bp RsrII-SphI fragment of pBAC/PRRSV/FL was subcloned into the RsrII- and SphI-digested pRS2 vector, resulting in pRS/RSS.

(i) pBAC/PRRSV/RepLuc MB: The 6906-bp BsrGI (T4 DNA polymerase-treated)-MluI (T4 DNA polymerase-treated) fragment of pRS/RSS was ligated with the 2325-bp AatII (T4 DNA polymerase-treated)-NsiI (T4 DNA polymerase-treated) fragment of pBAC^{SP6}/JVFLx/LUC/XbaI (Yun et al., J. Virol. 77: 6450-6465, 2003), which contains the expression cassette containing the LUC gene driven by the internal ribosome entry site (IRES) of encephalomyocarditis virus (EMCV). This produced the pRS/MB construct. The 6473-bp RsrII-NotI fragment of pRS/MB was then ligated with the 17584-bp RsrII-NotI fragment of pBAC/PRRSV/FL, resulting in the construction of pBAC/PRRSV/RepLuc MB represented by SEQ. ID. No 35.

(ii) pBAC/PRRSV/RepLuc DI: The 5301-bp EcoRV-BbvCI (T4 DNA polymerase-treated) fragment of pRS/RSS was ligated with the 2325-bp AatII (T4 DNA polymerase-treated)-NsiI (T4 DNA polymerase-treated) fragment of pBAC^{SP6}/JVFLx/LUC/XbaI, resulting in pRS/DI. The 4865-bp RsrII-NotI fragment of pRS/DI was then ligated with the 17584-bp RsrII-NotI fragment of pBAC/PRRSV/FL, resulting in the construction of pBAC/PRRSV/RepLuc DI represented by SEQ. ID. No 36.

(iii) pBAC/PRRSV/RepLuc ME: The 3470-bp RsrII-BsrGI fragment of pBAC/PRRSV/RepLuc DI was ligated with two fragments of the pBAC/PRRSV/RepLuc MB (the 4666-bp BsrGI-XbaI and 15370-bp XbaI-RsrII fragments), resulting in the construction of pBAC/PRRSV/RepLuc ME represented by SEQ. ID. No 37.

(iv) pBAC/PRRSV/RepLuc S1-S8: Eight fragments (S1 to S8) of pBAC/PRRSV/FL were first PCR-amplified using PRdiR (SEQ. ID. No 38) as the reverse primer and the PRs1 (SEQ. ID. No 39), PRs2 (SEQ. ID. No 40), PRs3 (SEQ. ID. No 41), PRs4 (SEQ. ID. No 42), PRs5 (SEQ. ID. No 43), PRs6 (SEQ. ID. No 44), PRs7 (SEQ. ID. No 45), and PRs8 (SEQ. ID. No 46) primers as the forward primers, respectively. To facilitate the cloning steps, these eight amplicons were first subcloned. The 5071-bp NotI-EcoRV fragment of pRS/RSS and the 2325-bp AatII (T4 DNA polymerase-treated)-NsiI fragment of pBAC^{SP6}/JVFLx/LUC/XbaI were then ligated with the 286, 336, 386, 436, 486, 536, 586, and 986-bp PstI-NotI fragments of the S1-S8 amplicons to produce pRS/RepLuc S1-S8, respectively. The 17584-bp NotI-RsrII fragment of pBAC/PRRSV/FL was subsequently ligated with the 4922, 4972, 5022, 5072, 5122, 5172, 5222, and 5622-bp RsrII-NotI fragments of the pRS/RepLuc S1-S8 plasmids to obtain the pBAC/PRRSV/RepLuc S1-S8 plasmids represented by SEQ. ID. No 47-No 54, respectively.

(v) pBAC/PRRSV/FL/IRES-EGFP and pBAC/PRRSV/FL/N^{pro}-EGFP: To generate pBAC/PRRSV/FL/IRES-EGFP, fragment I was synthesized by PCR-amplification of pBAC^{SP6}/JVFLx/GFP/XbaI^{MBN} (Yun et al., J. Virol. 77: 6450-6465, 2003) with the O7-5 (SEQ. ID. No 55)+O7-6 (SEQ. ID. No 56) primers. Fragment II was obtained by PCR-amplification of pBAC/PRRSV/FL with the PRs8+PRdiR primers. The 748-bp XbaI-HindIII (T4 DNA polymerase-treated) portion of fragment I and the 992-bp PstI (T4 DNA polymerase-treated)-NotI portion of fragment II were ligated with the 2736-bp XbaI-NotI fragment of pRS2 vector, resulting in pRS/DIs8. Fragment III was synthesized by PCR-amplification of pBAC/PRRSV/FL with the PR40+O7-2 (SEQ. ID. No 57) primers. The 3697-bp NcoI-BsrGI fragment of pRS/DIs8 was then ligated with the 1271-bp PciI-BglII (T4 DNA polymerase-treated) portion of the fragment III and the 1378-bp AatII (T4 DNA polymerase-treated)-BsrGI fragment of pBAC^{SP6}/JVFLx/GFP/XbaI^{MBN} (Yun et al., J. Virol. 77: 6450-6465, 2003), leading to pRS/DIs88. Thereafter, the 3122-bp MluI-NotI fragment of pRS/DIs88 was ligated with the two fragments of pBAC/PRRSV/FL (the 17584-bp NotI-RsrII and the 4348-bp RsrII-MluI fragments) to create pBAC/PRRSV/FL/IRES-EGFP represented by SEQ. ID. No 58.

To generate pBAC/PRRSV/FL/N^{pro}-EGFP, one fragment each from pBAC/PRRSV/FL and pBAC/NADLJiv90- (G1 and G2) was first amplified by PCR with the O7-1 (SEQ. ID. No 59)+O7-2 and O7-3 (SEQ. ID. No 60)+O7-4 (SEQ. ID. No 61) primers, respectively. The 731-bp XhoI-BglII fragment of the G1 amplicons and the 510-bp BglII-XbaI fragment of the G2 amplicons were then ligated with the 2743-bp XhoI-XbaI fragment of pRS2 vector, resulting in pRS/cNpro. Subsequently, one fragment each from pEGFP-C1 (Clontech Laboratories, Inc., Palo Alto, Calif.) and pBAC/PRRSV/FL (G3 and G4) was obtained by PCR with the O7-5+O7-6 and O7-7 (SEQ. ID. No 62)+O7-8 (SEQ. ID. No 63) primers, respectively. The 748-bp XbaI-HindIII fragment of the G3 amplicons and the 495-bp HindIII-NcoI fragment of the G4 amplicons were ligated with the 3926-bp XbaI-NcoI fragment of pRS/cNpro to generate the pRS/cNpro-EGFP3n subclone construct. Finally, the 2470-bp MluI-NotI fragment of pRS/cNpro-EGFP3n was ligated with two fragments of pBAC/PRRSV/FL (the 17584-bp NotI-RsrII and the 4348-bp RsrII-MluI fragments), to construct pBAC/PRRSV/FL/N^{pro}-EGFP represented by SEQ. ID. No 64.

The present inventors sought to construct a panel of self-replicating self-limited PRRSV viral replicons by using pBAC/PRRSV/FL. The inventors initially constructed a set of three viral replicons, designated as pBAC/PRRSV/RepLuc MB, pBAC/PRRSV/RepLuc ME, and pBAC/PRRSV/RepLuc DI, which have large internal deletions of nt 12714-14194, nt 12163-14194, and nt 12163-15252, respectively (FIG. 10). To facilitate the monitoring of viral replication, the present inventors also inserted at the site of each deletion the expression cassette containing the EMCV IRES-driven LUC gene. LUC was chosen as the reporter since its expression is easy to monitor in a highly quantitative and sensitive manner. The present inventors then examined whether the viral replicon RNAs derived from the three cDNA templates were replication-competent by monitoring the induction of the LUC gene after their transfection in BHK-21 cells. Particularly, electroporated cells grown in a 6-well plate were washed with PBS twice, lysed with 200 μl per well of cell lysis buffer (Promega, Co., Madison, Wis.) and 5 μl of the lysate was used to estimate LUC activity by adding 50 μl of LUC substrate and subsequently measuring its activity for 20 sec in a TD-20/20 luminometer (Promega). The experiments were conducted in triplicate; mean values are presented.

As a result, the LUC activities 6 hr after transfection with PRRSV/RepLuc MB or PRRSV/RepLuc ME RNAs were 9.95×10² and 9.72×10² relative light units (RLU), respectively (FIG. 11) and were dramatically increased at 18 and 24 hr post-transfection to 1.12-1.49×10⁴ and 2.12-2.45×10⁴ RLU, respectively. These activities were maintained until 48 hr post-transfection (FIG. 11), after which they gradually decreased due to the cell death induced by the replication of these viral replicons and the lack of cell-to-cell spread. In BHK-21 cells transfected with PRRSV/RepLuc DI RNA, the initial LUC activity at 6 hr post-transfection was similar to that of the other two (9.81×10² RLU) but then gradually decreased over time to 3.60 RLU at 72 hr post-transfection, which is at the level of the background luminescence from the mock-transfected cells (FIG. 11). Thus, PRRSV/RepLuc MB and PRRSV/RepLuc ME viral replicon RNAs are competent in replication but PRRSV/RepLuc DI RNA is not.

To determine the location of the minimal cis-acting element required for viral replication at the 3′ end of the viral genome, the present inventors constructed the eight viral replicons pBAC/PRRSV/RepLuc S1 to S8 by systematically deleting additional sequences towards the 3′ end of pBAC/PRRSV/RepLuc ME (FIG. 10). Of the eight viral replicons, only PRRSV/RepLuc S8 RNAs were replication-competent (FIG. 11). Thus, the 911 nucleotides from the 3′ end of the viral genome contain the minimal cis-acting element required for viral replication.

Immunostaining of the ORF7 protein showed that it was expressed in BHK-21 cells transfected with the replication-competent PRRSV/RepLuc S8, PRRSV/RepLuc MB and PRRSV/RepLuc ME RNAs but not in cells transfected with the replication-incompetent viral replicons. Previous work with a coronavirus system has indicated that the N protein, which is equivalent to the PRRSV ORF7 protein, may play an as yet unspecified role in coronavirus gene expression (Thiel et al., J. Virol. 77: 9790-9798, 2003). Thus, the present inventors examined whether ORF7 protein expression is required for the replication of the replicons the present inventors constructed. This question was addressed by two different approaches. In the first approach, the present inventors constructed the PRRSV ORF7 expression vector pSinRep19/PRRSV ORF7 by using the Sindbis virus-based pSinRep19 (Agapov et al., Proc. Natl. Acad. Sci. 95: 12989-12994, 1998), which contains a double subgenomic promoter (26S promoter, one for the foreign gene of interest and another for the PAC gene that enables selection with the antibiotic puromycin) (FIG. 12A). A BHK-21 cell line that stably expresses PRRSV ORF7 was established by transfecting BHK-21 cells with in vitro transcribed SinRep19/PRRSV ORF7 RNAs followed by selection with puromycin.

Particularly, the present inventors first constructed the PRRSV ORF7 expression vector pSinRep19/PRRSV ORF7 by using the Sindbis virus-based pSinRep19 (Agapov et al., Proc. Natl. Acad. Sci. 95: 12989-12994, 1998). Thus, a fragment from pBAC/PRRSV/FL was amplified by PCR with the PRorf7F (SEQ. ID. No 65)+PRorf7R (SEQ. ID. No 66) primers. The 381-bp XbaI-PmeI portion of these amplicons was then ligated with the 10852-bp XbaI-PmlI fragment of pSinRep19, resulting in pSinRep19/PRRSV ORF7 represented by SEQ. ID. No 67. pSinRep19/PRRSV ORF7 or pSinRep19 was linearized by digestion with XhoI and used as a template for in vitro transcription using the SP6 RNA polymerase, as described above. The SinRep19/PRRSV ORF7 RNA or SinRep19 RNA transcripts were then transfected into BHK-21 cells.

After transfection, the cells were seeded for ≈24 hr, after which the medium was replaced with fresh complete media containing 10 μg/ml puromycin for selection (Sigma-Aldrich Co., St. Louis, Mo.). Thereafter, the cells were maintained in the presence of puromycin and passaged or frozen.

Immunostaining of ORF7 showed that it was expressed throughout the cytoplasm as well as in the nucleolus (FIG. 12B). This is consistent with previous reports (Rowland et al., Virology 316: 135-145, 2003; Rowland and Yoo, Virus Res. 95: 23-33, 2003; Yoo et al., J. Virol. 77: 12173-12183, 2003). The SinRep19/PRRSV ORF7-expressing cells or control SinRep19-expressing cells were then transfected with PRRSV/RepLuc S1-S8 and LUC activity was monitored. Naive BHK-21 cells were also transfected in parallel with the same eight viral replicon RNAs. The PRRSV/RepLuc S8 viral replicon continued to be replication-competent in the presence of the ectopic expression of the ORF7 protein while the PRRSV/RepLuc S1-S7 viral replicons remained replication-incompetent (FIG. 12C). Identical results were obtained when all PRRSV viral replicon RNAs were co-transfected with SinRep19/PRRSV ORF7 RNAs.

In the second approach, the present inventors co-transfected BHK-21 cells with the infectious PRRSV viral RNA transcribed from pBAC/PRRSV/FL and each of the eight PRRSV viral replicon RNAs. As a result, the replication-incompetent viral replicon RNAs remained replication-incompetent while PRRSV/RepLuc S8 viral replicon RNA continued to be replication-competent (FIG. 12D). Thus, the replication-incompetent viral replicon RNAs (PRRSV/RepLuc S1-S7) are still replication-incompetent when the ORF7 protein is expressed in trans. This is consistent with the previous work that showed that expression of the structural proteins of EAV is not necessary for the replication of its genomic RNA (Molenkamp et al., J. Gen. Virol. 81: 2491-2496, 2000). Thus, PRRSV replication is not dependent on ORF7 protein expression.

### Example 10

### Heterologous Gene Expression by Infectious PRRSV cDNA/Recombinant Viruses

### <10-1> Analysis of Reporter Gene Expression

The present inventors sought to produce recombinant infectious PRRSV viruses that express the EGFP reporter gene upon infection. To do this, pBAC/PRRSV/FL was engineered in two ways. First, the present inventors inserted the EMCV IRES-driven EGFP expression cassette immediately downstream of the first 33 nucleotides of the ORF7 coding region that include a short PRRSV transcription-regulating sequence, and followed it by the cis-acting element in the 3′ 911 nucleotides of the viral genome (FIG. 13A). The EGFP gene in the resulting pBAC/PRRSV/FL/IRES-EGFP construct is driven by the EMCV IRES and its expression is dependent on viral replication. Second, the autoprotease N^{pro} gene of bovine viral diarrhea virus was fused adjacent to the N-terminus of the EGFP gene, so that the correct N-terminus of the EGFP protein is created by N^{pro} cleavage (FIG. 13A). The fused N^{pro}-EGFP gene was then inserted downstream of the first 33 nucleotides of the ORF7 coding region that include a short PRRSV transcription-regulating sequence that is required for the synthesis of the subgenomic mRNA of the N^{pro}-EGFP gene. This resulted in the pBAC/PRRSV/FL/N^{pro}-EGFP construct. In both constructs, the EGFP expression cassette was inserted downstream of the first 33 nucleotides of the ORF7 coding region because this region may be involved in the gene expression and regulation of its own subgenomic mRNA. In both cases, the insertion did not alter the infectivity of the synthetic RNA transcripts.

EGFP expression was then assessed by transfecting naïve BHK-21 cells with the PRRSV/FL, PRRSV/FL/IRES-EGFP, or PRRSV/FL/N^{pro}-EGFP RNAs that had been transcribed in vitro and examining them by confocal microscopy. To determine EGFP expression, electroporated cells grown in a 4-well chamber slide were washed twice with PBS and fixed in PBS containing 0.37% (v/v) formaldehyde for 30 min at 25° C. The cells were then mounted with 0.2 ml 80% glycerol and viewed with a Zeiss Axioskop confocal microscope with a 63× objective and a Bio-Rad MRC 1024. The images were analyzed by using LaserSharp software. As a result, no fluorescence was observed in the PRRSV/FL RNA-transfected or mock-transfected cells but the other two cell types showed green fluorescence (FIG. 13B). The fluorescence was in the nucleus and the cytoplasm because EGFP (≈30 kDa) is small enough to permit diffusion between the nucleus and the cytoplasm. All three RNA-transfected cell types also expressed the ORF7 protein (FIG. 13B). Similar observations were made when MARC-145 cells were transfected instead.

The production of recombinant EGFP-expressing PRRSV viruses was estimated by measuring (at the indicated time points) the viral titers in the culture supernatants of BHK-21 or MARC-145 cells transfected with the synthetic RNA transcripts (PRRSV/FL/IRES-EGFP and PRRSV/FL/N^{pro}-EGFP RNAs). Production of the EGFP-expressing PRRSV viruses was monitored by infecting naïve MARC-145 cells and subsequently counting the green focus-forming units per ml (GFU/ml). Several experiments showed that transfection of MARC-145 cells with PRRSV/FL/IRES-EGFP RNAs generated 1.2×10³, 1.0×10⁴, and 9.6×10⁴ GFU/ml at 24, 48, and 72 hr post-transfection, respectively (FIG. 13C). Similar virus titers were produced by PRRSV/FL/N^{pro}-EGFP RNA-transfected MARC-145 cells (FIG. 13C). In addition, at 24 hr post-transfection, BHK-21 cells transfected with the EGFP-expressing PRRSV RNAs produced slightly higher virus titers (4.1-5.5×10⁴ GFU/ml) than the MARC-145 cells due to the higher transfection efficiency of BHK-21 cells. Moreover, unlike MARC-145 cells, the BHK-21-generated virus titers gradually decreased to 6.4-7.8×10² GFU/ml at 96 hr post-transfection due to a lack of viral spread and cell death of the synthetic RNA-containing cells. Thus, the present inventors were able to produce recombinant PRRSVs encoding the EGFP gene as a reporter, which reveals the possibility of utilizing infectious PRRSV cDNA/recombinant viruses as heterologous gene expression vectors.

### Example 11

### Importance of the PRRSV 5′ End Nucleotide Sequence ¹ATGACGT⁷ in RNA Replication; Serial Nucleotide Deletions of the PRRSV 5′ End in Genomic RNAs Decrease or Completely Abolish Their Infectivity, Which Appears to be Restored by the Acquisition of Novel AT-Rich 5′ Sequences

8 constructs (pBAC/PRRSV/FL/Δnt1, pBAC/PRRSV/FL/Δnt3, pBAC/PRRSV/FL/Δnt5, pBAC/PRRSV/FL/Δnt7, pBAC/PRRSV/FL/Δnt9, pBAC/PRRSV/FL/Δnt11, pBAC/PRRSV/FL/Δnt13, and pBAC/PRRSV/FL/Δnt15) were constructed to have deletions of 1, 3, 5, 7, 9, 11, 13, and 15 nucleotides from the 5′-terminus of the viral genome in pBAC/PRRSV/FL. The construction method is as follows (FIG. 14). Each deletion was prepared by PCR-based mutagenesis. Two fragments from pBAC/PRRSV/FL/Δnt1 were PCR-amplified with PRfor (SEQ. ID. No 69)+1R (SEQ. ID. No 70) and 1F (SEQ. ID. No 71)+PRrev (SEQ. ID. No 72) primer pairs. Those two fragments were fused with each other by a second round of PCR using PRfor and PRrev primers. The 451-bp PacI-SacI fragment of the resulting amplicon was ligated with the 9350-bp SacI-RsrII and 13237-bp RsrII-PacI fragments of pBAC/PRRSV/FL, resulting in the construction of the construct pBAC/PRRSV/FL/Δnt1. Likewise, for the construction of 7 other constructs (pBAC/PRRSV/FL/Δnt3, pBAC/PRRSV/FL/Δnt5, pBAC/PRRSV/FL/Δnt7, pBAC/PRRSV/FL/Δnt9, pBAC/PRRSV/FL/Δnt11, pBAC/PRRSV/FL/Δnt13 and pBAC/PRRSV/FL/Δnt15), each of the first two fragments were PCR-amplified by using PRfor+3R (SEQ. ID. No 73)/3F (SEQ. ID. No 74)+PRrev, PRfor+SR (SEQ. ID. No 75)/5F (SEQ. ID. No 76)+PRrev, PRfor+7R (SEQ. ID. No 77)/7F (SEQ. ID. No 78)+PRrev, PRfor+9R (SEQ. ID. No 79)/9F (SEQ. ID. No 80)+PRrev, PRfor+11R (SEQ. ID. No 81)/11F (SEQ. ID. No 82)+PRrev, PRfor+13R (SEQ. ID. No 83)/13F (SEQ. ID. No 84)+PRrev, and PRfor+15R (SEQ. ID. No 85)/15F (SEQ. ID. No 86)+PRrev primer sets, respectively. The resultant two fragments were ligated with each other by a second round of PCR using PRfor and PRrev primers, as described earlier.

The present inventors determined the specific infectivity of the synthetic RNA transcripts derived from each truncated mutant cDNA template. Particularly, deletions of 1 (pBAC/PRRSV/FL/Δnt1) and 3 (pBAC/PRRSV/FL/Δnt3) nucleotides at the utmost 5′ end were equally decreased their infectivity by 15-fold (1.2-3.1×10⁴ PFU/μg), as compared to the wild-type infectious cDNA pBAC/PRRSV/FL (4.4-5.2×10⁵ PFU/μg). Moreover, deletions of 5 (pBAC/PRRSV/FL/Δnt5) and 7 (pBAC/PRRSV/FL/Δnt7) nucleotides drastically decreased the infectivity to 1.0-1.8×10² PFU/μg and 8.6-9.3×10² PFU/μg, which were ≈3500-fold and ≈550-fold lower than that of the wild type, respectively (FIG. 15). Interestingly, the infectivity of the synthetic RNAs containing the 7-nucleotide deletion was generally ≈6-fold higher than that containing the 5-nucleotide deletion.

In addition, the present inventors also examined the morphology of plaques and foci recognized by immunostaining with a mouse anti-ORF7 Mab. In the cells transfected with the synthetic RNAs derived from pBAC/PRRSV/FL/Δnt1 and pBAC/PRRSV/FL/Δnt3, the present inventors observed a relatively homogeneous population of large plaques and foci, as seen in the cells transfected with the wild-type infectious RNAs (FIG. 15). In the cells transfected with the synthetic RNAs derived from pBAC/PRRSV/FL/Δnt5 and pBAC/PRRSV/FL/Δnt7, on the other hand, a relatively heterogeneous population of smaller plaques and foci were recognized (FIG. 15). Neither infectivity nor foci/plaques were detectable with synthetic RNAs containing more than 9-nucleotide deletions at the utmost 5′end (FIG. 15). Thus, serial nucleotide deletions at the 5′ end of PRRSV genomic RNA reduced or completely abolished the infectivity.

It should be noted that two mutants harboring the deletion of 5 and 7 nucleotides produced plaques of heterogeneous sizes, indicating some instability. This is more evident when supernatants harvested from the transfected cells were passaged once on naive MARC-145 cells. These passaged pseudorevertants derived from the mutant cDNAs containing 1-, 3-, 5-, and 7-nucleotide deletions produced similar amounts of the PRRSV Nsp1a protein upon infection with the same amount of the viruses, as compared to the wild-type virus (FIG. 16). Furthermore, their growth kinetics (FIG. 17) and plaque morphology (FIG. 18) were very similar, with a relatively homogeneous population of large plaques observed in the cells infected with each of these pseudorevertants, including the 5- and 7-nucleotide deletion mutants. The nucleotide sequence at the utmost 5′ end region of all these pseudorevertants was determined by 5′ RACE analysis, cloning of RT-PCR amplicons, and sequencing of a number of the independently picked clones containing the insert.

As summarized in FIG. 19, 33 of 42 independent clones obtained from the pBAC/PRRSV/FL/Δnt1-derived pseudorevertants appeared to be converted to the wild-type virus by the acquisition of one A nucleotide at the site of the deletion. The remainder of the 9 clones had the same sequence as pBAC/PRRSV/FL/Δnt1 (FIG. 19). In case of the pBAC/PRRSV/FL/Δnt3-derived pseudorevertants, 32 out of 57 independent clones had acquired three nucleotides (ATG) at the utmost 5′ end, which render the sequence identical to the wild-type virus. Twenty-one clones contained an insertion of 4 (TATG) while four clones appeared to contain an insertion of 3 (AAG) nucleotides at the deletion site (FIG. 19). Interestingly, the pBAC/PRRSV/FL/Δnt5-derived pseudorevertants were found to have a deletion of the 5′-end single G nucleotide, the first nucleotide in this mutant construct. Moreover, in 39 of 49 independent clones there was an insertion of 6 novel (ATTATA) nucleotides at the deletion site while 10 clones contained a 7-nucleotide insertion (TATTATA) (FIG. 19). In case of the pBAC/PRRSV/FL/Δnt7-derived pseudorevertants, the acquisition of novel 5′ end sequences was found to be more heterogeneous than the mutants described above. Specifically, a majority of the sequenced clones (28/48 and 9/48 clones) had an insertion of 7 (ATTATAT) and 8 (TATTATAT) nucleotides at the site of the deletion, respectively, and appeared to be identical to two of the pBAC/PRRSV/FL/Δnt5-derived pseudorevertants. In addition, 4 independent clones had 8 novel nucleotides (TATCATAT) inserted at the deletion site while 2 further clones had the 8-nucleotide sequence ATTTATAT inserted at this site. In addition, 3 and 2 independent clones appeared to have 5 and 6 AT repeats at the site of the deletion, respectively (FIG. 19). Overall, these pseudorevertants appeared to have acquired novel 5′ sequences of various sizes, composed mainly of A and T bases.

To address the importance of these novel 5′ sequences, the present inventors reconstructed 8 derivatives of the PRRSV 5′-end truncated mutants with all of these novel sequences and determined the specific infectivities of their RNA transcripts (FIG. 20). As shown in FIG. 19, pBAC/PRRSV/FL/Δnt3/Rev2 and pBAC/PRRSV/FL/Δnt3/Rev3 were constructed by adding 4 (TATG) and 3 (AAG) nucleotides to starting point of the viral genome of pBAC/PRRSV/FL/Δnt3. 6 additional constructs, pBAC/PRRSV/FL/Δnt7/Rev1˜6, were constructed by adding 7 (ATTATAT), 8 (TATTATAT, TATCATAT or ATTTATAT), 10 (ATATATATAT), and 12 (ATATATATATAT) nucleotides to starting point of viral genome in pBAC/PRRSV/FL/Δnt7. The method for the construction of 8 derivatives having novel PRRSV 5′ ends is the same as the construction method for pBAC/PRRSV/FL/Δnt1-Δnt15 described earlier. Briefly, to construct 8 derivatives, the 451-bp PacI-SacI fragment bearing novel PRRSV 5′ end sequences was PCR amplified with PRfor and PRrev primers by using clones having the novel PRRSV 5′ end sequence used for the sequencing analyses above as templates. The 451-bp PacI-SacI fragments of the resulting amplicons were ligated with the 9350-bp SacI-RsrII and 13237-bp RsrII-PacI fragments of pBAC/PRRSV/FL, resulting in 8 corresponding derivatives.

In all reconstructed cases, their specific infectivities were increased to a level similar to that of the wild type (FIG. 20). Among all of the pseudorevertants, the present inventors found three cases that were not reconstructed, since the resulting mutations were identical to either the wild type (PRRSV/FL/Δnt1/Rev1 and PRRSV/FL/Δnt3/Rev1) or the original truncated mutant (PRRSV/FL/Δnt1/Rev2). According to plaque/focus morphology, the cells transfected with four synthetic RNAs (derived from pBAC/PRRSV/FL/Δnt3/Rev2, pBAC/PRRSV/FL/Δnt7/Rev1, pBAC/PRRSV/FL/Δnt7/Rev2, and pBAC/PRRSV/FL/Δnt7/Rev6) formed a homogeneous population of large plaques/foci, as seen with the wild-type infectious cDNA. In addition, the present inventors also observed a homogeneous population of medium (pBAC/PRRSV/FL/Δnt3/Rev3 and pBAC/PRRSV/FL/Δnt7/Rev3) and small (pBAC/PRRSV/FL/Δnt7/Rev4 and pBAC/PRRSV/FL/Δnt7/Rev5) sized plaques/foci (FIG. 20). These results demonstrate that the addition of novel AT-rich sequences to the utmost 5′ end of the PRRSV 5′ deletion mutants, but not changes elsewhere in their genomes, allows efficient PRRSV replication.

The present invention elucidates the importance of the PRRSV 5′ end nucleotide sequence ¹ATGACGT⁷ in RNA replication. Several novel AT-rich PRRSV 5′ sequences detected in pseudorevertants were able to functionally replace the deleted ¹ATGACGT⁷. Although the functional role of these novel sequences is not completely understood, the complementary sequence of each of these novel 5′ sequences at the utmost 3′ end of negative-sense RNA is predicted to be involved in the initiation of positive-sense RNA synthesis. For some positive-sense RNA viruses, only minimal cis-acting sequences at the 3′ end of negative-sense RNAs are essential for positive-sense RNA synthesis (Ball L. A., Proc. Natl. Acad. Sci. USA 91: 12443-12447, 1994; Guan H. et al., RNA 3: 1401-1412, 1997; Pugachev K. V. and Frey T. K., J. Virol. 72: 641-650, 1998).

It is interesting to speculate on the origin of these novel 5′ AT-rich sequences and the molecular mechanism of how their insertion at the very beginning of the viral genome. Based on the heterogeneity and size of these novel 5′ sequences, it is likely that they are acquired from cellular RNAs in the process of recombination involving template switching, during either negative- or positive-sense RNA synthesis, as has been described for pestivirus (Meyers G. and Thiel H. J., Adv. Virus Res. 47: 53-118, 1996) and poliovirus (Kirkegaard K. and Baltimore D., Cell 47: 433-443, 1986). It is less likely, although not impossible, that these sequences may be derived from the viral genome. Understanding of this issue may provide new information on RNA replication of arteriviruses.

### INDUSTRIAL APPLICABILITY

As explained hereinbefore, the infectious PRRSV cDNA clone and its derivative cDNAs of the present invention can be effectively used not only for studies on molecular biological mechanisms involved in replication, transcription and translation of PRRSV, but also for the development of a therapeutic agent, a vaccine, a diagnostic agent, and a diagnostic kit for PRRS. The infectious PRRSV cDNA of the present invention can also be used as a PRRSV vector for the expression of a heterologous foreign gene in cells, in vivo and in vitro, for the DNA immunization and for the temporary gene therapy.

### [Sequence List Text]

The nucleotide sequences represented by SEQ. ID. No 1˜17 are the primer sequences used for sequencing the complete full-length genomic RNA of PL97-1/LP1 and for the construction of the infectious cDNA for PL97-1/LP1 virus,

The nucleotide sequence represented by SEQ. ID. No 1.8 is the complete full-length nucleotide sequence of PL97-1/LP1,

The nucleotide sequences represented by SEQ. ID. No 19˜21 are the primer sequences used for the synthesis of Fr4c amplicon during the construction of the infectious cDNA for PL97-1/LP1 virus,

The nucleotide sequences represented by SEQ. ID. No 22˜24 are the primer sequences used for the introduction of SP6 promoter sequence during the construction of the infectious cDNA for PL97-1/LP1 virus,

The nucleotide sequences represented by SEQ. ID. No 25˜26 are the sequences used for engineering a run-off site during the construction of the infectious cDNA for PL97-1/LP1 virus,

The nucleotide sequence represented by SEQ. ID. No 27 is the sequence of the infectious cDNA pBAC/PRRSV/FL in which the full-length cDNA of PRRSV PL97-1/LP1 virus genome is cloned,

The nucleotide sequences represented by SEQ. ID. No 28˜29 are the primer sequences used for PCR-amplification of a region containing a silence point mutation in Example 6,

The nucleotide sequence represented by SEQ. ID. No 30 is the sequence of pBAC/PRRSV/FLgm constructed by the method described in Example 6,

The nucleotide sequence represented by SEQ. ID. No 31 is the primer sequence used for the confirmation of a silent point mutation in Example 6,

The nucleotide sequence represented by SEQ. ID. No 32 is the sequence of pBAC/PRRSV/FLnop(A) in which the poly (A) tail, in Example 8, is deleted,

The nucleotide sequences represented by SEQ. ID. No 33˜34 are the primer sequences for the construction of pBAC/PRRSV/FLnop(A) of Example 8,

The nucleotide sequences represented by SEQ. ID. No 35˜37 are the sequences of PRRSV viral replicons constructed by the method described in Example 9,

The nucleotide sequences represented by SEQ. ID. No 38˜46 are the primer sequences used for the construction of pBAC/PRRSV/RepLuc S1-S8 of Example 9,

The nucleotide sequences represented by SEQ. ID. No 47˜54 are the nucleotide sequences of pBAC/PRRSV/RepLuc S1-S8 constructed in Example 9,

The nucleotide sequences represented by SEQ. ID. No 55˜57 are the primer sequences used for the construction of pBAC/PRRSV/FL/IRES-EGFP of Example 9,

The nucleotide sequence represented by SEQ. ID. No 58 is the sequence of pBAC/PRRSV/FL/IRES-EGFP constructed in Example 9,

The nucleotide sequences represented by SEQ. ID. No 59˜63 are the primer sequences used for the construction of pBAC/PRRSV/FL/N^{pro}-EGFP of Example 9,

The nucleotide sequence represented by SEQ. ID. No 64 is the sequence of pBAC/PRRSV/FL/N^{pro}-EGFP constructed in Example 9,

The nucleotide sequences represented by SEQ. ID. No 65˜66 are the primer sequences used for the construction of pSinRep19/PRRSV ORF7 vector,

The nucleotide sequence represented by SEQ. ID. No 67 is the sequence of pSinRep19/PRRSV ORF7 constructed in Example 9,

The nucleotide sequence represented by SEQ. ID. No 68 is the nucleotide sequence of a cDNA harboring SP6 promoter and run-off site corresponding to the full-length PRRSV RNA,

The nucleotide sequences represented by SEQ. ID. No 69˜86 are the primer sequences used for the construction of PRRSV 5′ nucleotide deletion mutants and for the reconstruction of infectious pseudorevertants of Example 11.

1. A genetically stable full-length infectious PRRSV cDNA corresponding to the complete full-length PRRSV genomic RNA.

2. The PRRSV cDNA according to claim 1, wherein the cDNA includes a promoter upstream of the 5′end of PRRSV genomic RNA and a nucleotide sequence corresponding to a restriction endonuclease recognition site downstream of the 3′ end of PRRSV genomic RNA as a run-off site.

3. The PRRSV cDNA according to claim 2, wherein the promoter is a SP6 promoter.

4. The PRRSV cDNA according to claim 2, wherein the nucleotide sequence corresponding to a restriction endonuclease recognition site does not exist in the PRRSV genomic RNA.

5. The PRRSV cDNA according to claim 2, wherein the nucleotide sequence corresponding to a restriction endonuclease recognition site is selected from a group consisting of AclI^{MBN}, AclI, NotI and SdaI.

6. The PRRSV cDNA according to claim 2, wherein the cDNA has a SP6 promoter and is composed of nucleotide sequence represented by SEQ. ID. No 68.

7. A vector containing the PRRSV cDNA corresponding to the full-length PRRSV genomic RNA of claim 1.

8. The vector according to claim 7, wherein the vector uses BAC (bacterial artificial chromosome) as a parental vector.

9. The vector according to claim 7, wherein the vector has a SP6 promoter and is selected from a group consisting of pBAC/PRRSV/FL/AclI^{MBN}, pBAC/PRRSV/FL/AclI, pBAC/PRRSV/FL/NotI and pBAC/PRRSV/FL/SdaI containing the PRRSV cDNA represented by SEQ. ID. No 27.

10. The vector according to claim 7, wherein the vector is pBAC/PRRSV/FL represented by SEQ. ID. No 27 (Accession No; KCTC 10664BP).

11. An infectious PRRSV RNA transcript synthesized from the vector of claim 7.

12. The infectious PRRSV RNA transcript according to claim 11, wherein the virus-unrelated nucleotides at the 3′ end are removed.

13. The infectious PRRSV RNA transcript according to claim 12, wherein the virus-unrelated nucleotides at the 3′ end are removed by treating with mung bean nuclease.

14. A transfectant obtained by transfecting host cells with the PRRSV RNA transcript of claim 11.

15. The transfectant according to claim 14, wherein the host cells are all animal cells.

16. A synthetic PRRSV obtained by culturing the transfectant of claim 14.

17. The synthetic PRRSV according to claim 16, wherein the virus has a mutation introduced by engineering the mutation into the PRRSV cDNA.

18. A method for expressing heterologous genes by using the PRRSV cDNA comprising the following steps:
1) Preparing a recombinant PRRSV cDNA expression vector by inserting a heterologous gene into the infectious PRRSV cDNA vector of claim 7;
2) Preparing the PRRSV RNA transcript from the above recombinant PRRSV cDNA expression vector;
3) Preparing a transfectant by transfecting host cells with the above PRRSV RNA transcript; and
4) Expressing the inserted heterologous gene by culturing the transfectant cells.

19. The method according to claim 17, wherein the heterologous gene is inserted into the full-length infectious PRRSV cDNA.

20. The method according to claim 17, wherein the heterologous gene is inserted into PRRSV viral replicon cDNA's.

21. A diagnostic reagent containing components originated from the infectious PRRSV cDNA of claim 1.

22. A diagnostic reagent containing components originated from PRRSV viral replicon cDNAs of claim 7.

23. An anti-PRRSV vaccine containing components originated from the infectious PRRSV cDNA of claim 1.

24. A PRRSV cDNA deletion mutant in which 1-15 nucleotides from the 5′ end of the PRRSV cDNA of claim 1 are deleted.

25. The PRRSV cDNA according to claim 24, wherein the number of the deleted nucleotides is 1-7 and the PRRSV cDNA is infectious.

26. The PRRSV cDNA according to claim 24 wherein the infectivity is recovered by adding nucleotide sequences of various sizes to the 5′ end and the resultant PRRSV pseudorevertants.

27. The PRRSV pseudorevertants according to claim 26, wherein the revertants have an AT-rich nucleotide sequence.

28. The PRRSV pseudorevertants according to claim 27, wherein the AT-rich nucleotide sequence is selected from a group consisting of TATG, AAG, ATTATA, TATTATA, ATTATAT, TATTATAT, TATCATAT, ATATATATAT, ATATATATATAT and ATTTATAT.
